(19) United States
(12) Patent Application Publication
(10) Pub. No.: US 2003/0082538 A1
(43) Pub. Date: May 1, 2003

(54) Analysis of data from liquid chromatographic separation of DNA

(76) Inventors: Paul D. Taylor, Gilroy, CA (US); Elisa Yu, Fremont, CA (US)

Correspondence Address: JOHN F. BRADY, TRANSGENOMIC, INC., 2032 CONCOURSE DRIVE, SAN JOSE, CA, 95131, US

(21) Appl. No.: 09/873,645

(22) Filed: Jun. 4, 2001

Related U.S. Application Data
(60) Provisional application No. 60/209,231, filed on Jun. 2, 2000.
(60) Provisional application No. 60/231,396, filed on Sep. 8, 2000.
(60) Provisional application No. 60/253,491, filed on Nov. 27, 2000.
(60) Provisional application No. 60/255,274, filed on Dec. 12, 2000.
(60) Provisional application No. 60/257,421, filed on Dec. 23, 2000.
(60) Provisional application No. 60/282,008, filed on Apr. 5, 2001.
(60) Provisional application No. 60/282,070, filed on Apr. 5, 2001.

(51) Int. Cl. C12Q 1/68; G06F 19/00; G01N 33/48; G01N 33/50

(52) U.S. Cl. 435/6; 702/20

(57) ABSTRACT

In one aspect, the invention provides a computer implemented method for transforming a plurality of chromatographic elution profiles, wherein each profile is obtained from the separation of a mixture of homoduplex and heteroduplex molecules by Denaturing Matched Ion Polynucleotide Chromatography. The method preferably includes overlaying the profiles; adjusting the baseline by applying a slope factor to each detector response value so that all of the profiles share a common baseline; normalizing the heights of the peaks to a scale of 1; and shifting the profiles along the x-axis so that all of the profiles intersect at a pre-selected point. In another aspect, the invention provides a method for applying statistical criteria to the transformed profiles to determine whether they represent more that one group. In yet another aspect, the invention concerns methods for grouping the profiles based on their shapes. The methods for applying statistical test and for grouping the profiles include dividing the x-axis into equally spaced regions and determining the distribution of the profiles within the regions. The invention can be used for estimating the number of different single nucleotide polymorphisms in a plurality of test samples and for detecting the presence of a previously unknown polymorphisms. In other aspects, the invention concerns computer systems and computer readable media that can implement and store the data associated with the elution profiles and which can perform the transformation, statistical testing, and grouping processes.

### RELATIONSHIP TO CO-PENDING APPLICATIONS

This application is a regular U.S. Patent Application under 35 U.S.C. §111(a) and 35 C.F.R. §1.53(b) and claims priority from the following co-pending, commonly assigned provisional applications, each filed under 35 U.S.C. §111(b), each of which is incorporated herein in its entirety:

Serial No. 60/209,231 filed Jun. 2, 2000

Serial No. 60/231,396 filed Sep. 8, 2000

Serial No. 60/253,491 filed Nov. 27, 2000

Serial No. 60/255,274 filed Dec. 12, 2000

Serial No. 60/257,421 filed Dec. 23, 2000

Serial No. 60/282,008 filed Apr. 5, 2001

Serial No. 60/282,070 filed Apr. 5, 2001

### FIELD OF THE INVENTION

The present invention concerns detection of mutations in DNA. In particular, the invention concerns methods and devices for the analysis of elution profiles obtained from liquid chromatographic separation of double-stranded DNA.

### BACKGROUND OF THE INVENTION

The ability to detect mutations in double stranded polynucleotides, and especially in DNA fragments, is of great importance in medicine, as well as in the physical and social sciences. The Human Genome Project is providing an enormous amount of genetic information which is setting new criteria for evaluating the links between mutations and human disorders (Guyer et al., Proc. Natl. Acad. Sci. USA 92:10841 (1995)). The ultimate source of disease, for example, is described by genetic code that differs from wild type (Cotton, TIG 13:43 (1997)). Understanding the genetic basis of disease can be the starting point for a cure. Similarly, determination of differences in genetic code can provide powerful and perhaps definitive insights into the study of evolution and populations (Cooper, et. al., Human Genetics vol. 69:201 (1985)). Understanding these and other issues related to genetic coding is based on the ability to identify anomalies, i.e., mutations, in a DNA fragment relative to the wild type. A need exists, therefore, for a methodology to detect mutations in an accurate, reproducible and reliable manner.

DNA molecules are polymers comprising sub-units called deoxynucleotides. The four deoxynucleotides found in DNA comprise a common cyclic sugar, deoxyribose, which is covalently bonded to any of the four bases, adenine (a purine), guanine(a purine), cytosine (a pyrimidine), and thymine (a pyrimidine), hereinbelow referred to as A, G, C, and T respectively. A phosphate group links a 3′-hydroxyl of one deoxynucleotide with the 5′-hydroxyl of another deoxynucleotide to form a polymeric chain. In double stranded DNA, two strands are held together in a helical structure by hydrogen bonds between, what are called, complimentary bases. The complimentarity of bases is determined by their chemical structures. In double stranded DNA, each A pairs with a T and each G pairs with a C, i.e., a purine pairs with a pyrimidine. Ideally, DNA is replicated in exact copies by DNA polymerases during cell division in the human body or in other living organisms. DNA strands can also be replicated in vitro by means of the Polymerase Chain Reaction (PCR).

Sometimes, exact replication fails and an incorrect base pairing occurs, which after further replication of the new strand results in double stranded DNA offspring containing a heritable difference in the base sequence from that of the parent. Such heritable changes in base pair sequence are called mutations.

In the present invention, double stranded DNA is referred to as a duplex. When the base sequence of one strand is entirely complimentary to base sequence of the other strand, the duplex is called a homoduplex. When a duplex contains at least one base pair which is not complimentary, the duplex is called a heteroduplex. A heteroduplex duplex is formed during DNA replication when an error is made by a DNA polymerase enzyme and a non-complimentary base is added to a polynucleotide chain being replicated. Further replications of a heteroduplex will, ideally, produce homoduplexes which are heterozygous, i.e., these homoduplexes will have an altered sequence compared to the original parent DNA strand. When the parent DNA has the sequence which predominates in a natural population it is generally called the “wild type.”

Many different types of DNA mutations are known. Examples of DNA mutations include, but are not limited to, “point mutation” or “single base pair mutations” wherein an incorrect base pairing occurs. The most common point mutations comprise “transitions” wherein one purine or pyrimidine base is replaced for another and “transversions” wherein a purine is substituted for a pyrimidine (and visa versa). Point mutations also comprise mutations wherein a base is added or deleted from a DNA chain. Such “insertions” or “deletions” are also known as “frameshift mutations”. Although they occur with less frequency than point mutations, larger mutations affecting multiple base pairs can also occur and may be important. A more detailed discussion of mutations can be found in U.S. Pat. No. 5,459,039 to Modrich (1995), and U.S. Pat. No. 5,698,400 to Cotton (1997). These references and the references contained therein are incorporated in their entireties herein.

The sequence of base pairs in DNA codes for the production of proteins. In particular, a DNA sequence in the exon portion of a DNA chain codes for a corresponding amino acid sequence in a protein. Therefore, a mutation in a DNA sequence may result in an alteration in the amino acid sequence of a protein. Such an alteration in the amino acid sequence may be completely benign or may inactivate a protein or alter its function to be life threatening or fatal. On the other hand, mutations in an intron portion of a DNA chain would not be expected to have a biological effect since an intron section does not contain code for protein production. Nevertheless, mutation detection in an intron section may be important, for example, in a forensic investigation.

Detection of mutations is, therefore, of great interest and importance in diagnosing diseases, understanding the origins of disease and the development of potential treatments. Detection of mutations and identification of similarities or differences in DNA samples is also of critical importance in increasing the world food supply by developing diseases resistant and/or higher yielding crop strains, in forensic science, in the study of evolution and populations, and in scientific research in general (Guyer et al., Proc. Natl. Acad. Sci. USA 92:10841 (1995); Cotton, TIG 13:43 (1997)). These references and the references contained therein are incorporated in their entireties herein.

Alterations in a DNA sequence which are benign or have no negative consequences are sometimes called “polymorphisms”. In the present invention, any alterations in the DNA sequence, whether they have negative consequences or not, are called “mutations”. It is to be understood that the method of this invention has the capability to detect mutations regardless of biological effect or lack thereof. For the sake of simplicity, the term “mutation” will be used throughout to mean an alteration in the base sequence of a DNA strand compared to a reference strand. It is to be understood that in the context of this invention, the term “mutation” includes the term “polymorphism” or any other similar or equivalent term of art.

There exists a need for an accurate and reproducible analytical method for mutation detection which is easy to implement. Such a method, which can be automated and provide high throughput sample screening with a minimum of operator attention, is also highly desirable.

Analysis of DNA samples has historically been done using gel electrophoresis. Capillary electrophoresis has been used to separate and analyze mixtures of DNA. However, these methods cannot distinguish point mutations from homoduplexes having the same base pair length.

The “heteroduplex site separation temperature” is defined herein to mean, the temperature at which one or more base pairs denature, i.e., separate, at the site of base pair mismatch in a heteroduplex DNA fragment. Since at least one base pair in a heteroduplex is not complimentary, it takes less energy to separate the bases at that site compared to its fully complimentary base pair analog in a homoduplex. This results in the lower melting temperature of a heteroduplex compared to a homoduplex. The local denaturation creates, what is generally called, a “bubble” at the site of base pair mismatch. The bubble distorts the structure of a DNA fragment compared to a fully complimentary homoduplex of the same base pair length. This structural distortion under partially denaturing conditions has been used in the past to separate heteroduplexes and homoduplexes by denaturing gel electrophoresis and denaturing capillary electrophoresis. However, these techniques are operationally difficult to implement and require highly skilled personnel. In addition, the analyses are lengthy and require a great deal of set up time. A denaturing capillary gel electrophoresis analysis of a 90 base pair fragment takes more than 30 minutes and a denaturing gel electrophoresis analysis may take 5 hours or more. The long analysis time of the gel methodology is further exacerbated by the fact that the movement of DNA fragments in a gel is inversely proportional to the length of the fragments.

In addition to the deficiencies of denaturing gel methods mentioned above, these techniques are not always reproducible or accurate since the preparation of a gel and running an analysis is highly variable from one operator to another.

Recently, a chromatographic method called Matched Ion Polynucleotide Chromatography (MIPC), also referred to as ion-pair reverse phase high pressure liquid chromatography (IPRPHPLC), was introduced to effectively separate mixtures of double stranded polynucleotides, in general and DNA, in particular, wherein the separations are based on base pair length (Huber, et al., Chromatographia 37:653 (1993); Huber, et al., Anal. Biochem. 212:351 (1993); U.S. Pat. Nos. 5,585,236; 5,772,889; 5,972,222; 5,986,085; 5,997,742; 6,017,457; 6,030,527; 6,056,877; 6,066,258; 6,210,885; and U.S. patent application Ser. No. 09/129,105 filed Aug. 4, 1998).

The term “Matched Ion Polynucleotide Chromatography” as used herein is defined as a process for separating single and double stranded polynucleotides using non-polar separation media, wherein the process uses a counter-ion agent, and an organic solvent to release the polynucleotides from the separation media. MIPC separations are complete in less than 10 minutes, and frequently in less than 5 minutes. MIPC systems (WAVE® DNA Fragment Analysis System, Transgenomic, Inc. San Jose, Calif.) are equipped with computer controlled ovens which enclose the columns and column inlet areas.

As the use and understanding of MIPC developed it became apparent that when MIPC analyses were carried out at a partially denaturing temperature, i.e., a temperature sufficient to denature a heteroduplex at the site of base pair mismatch, homoduplexes could be separated from heteroduplexes having the same base pair length (Hayward-Lester, et al., Genome Research 5:494 (1995); Underhill, et al., Proc. Natl. Acad. Sci. USA 93:193 (1996); Doris, et al., DHPLC Workshop, Stanford University, (1997)). These references and the references contained therein are incorporated herein in their entireties. Thus, the use of DMIPC was applied to mutation detection (Underhill, et al., Genome Research 7:996 (1997); Liu, et al., Nucleic Acid Res., 26;1396 (1998)).

These chromatographic methods are generally used to detect whether or not a mutation exists in a test DNA fragment. In a typical experiment, a test fragment is hybridized with a wild type fragment and analyzed by DMIPC. If the test fragment contains a mutation, then the hybridization product includes both homoduplex and heteroduplex molecules. If no mutation is present, then the hybridization only produces homoduplex wild type molecules. The elution profile of the hybridized test fragment can be compared to a control in which a wild type fragment is hybridized to another wild type fragment. Any change in the elution profile (such as the appearance of new peaks or shoulders) between the hybridized test fragment and the control is assumed to be due to a mutation in the test fragment.

Single nucleotide polymorphisms (SNPs) are thought to be ideally suited as genetic markers for establishing genetic linkage and as indicators of genetic diseases (Landegre et al. Science 242:229-237 (1988)). In some cases a single SNP is responsible for a genetic disease. According to estimates the human genome may contain over 3 million SNPs. Due to their propensity they lend themselves to very high resolution genotyping. The SNP consortium, a joint effort of 10 major pharmaceutical companies, has announced the development of 300,000 SNP markers and their placement in the public domain by mid 2001. These facts increase the need for high throughput genotyping technologies.

A need exists to identify and optimize all the aspects of the MIPC methodology in order to minimize artifacts and remove ambiguity from the analysis of samples containing putative mutations.

Presently available systems include software and output devices that can display graphs showing chromatographic raw data consisting of detector response and time values. The analysis of multiple DNA samples leads to the generation of a plurality of chromatographic elution profiles. The data are usually displayed as stacked or overlayed elution profiles. Presently available systems are limited in their ability to analyze and interpret the large numbers of DMIPC elution profiles that are being generated.

There is a need for high-throughput, automated, and parallel processing of DNA samples for discovery and detection of mutations in multiple samples. There is a need for methods and devices for analyzing chromatographic elution profiles obtained by DMIPC and specifically for determining the relationship between the shape of elution profiles and the presence and identity of mutations such as SNPs.

### SUMMARY OF THE INVENTION

In one aspect, the invention concerns a computer implemented method for transforming a plurality of chromatographic elution profiles, wherein each profile is obtained from the separation of a DNA mixture by Denaturing Matched Ion Polynucleotide Chromatography, wherein each DNA mixture comprises homoduplex and heteroduplex molecules obtained from the hybridization of a sample DNA and its corresponding wild type DNA. The method includes the following steps:

a) overlaying the profiles on a coordinate system that includes a first axis associated with time values (i.e. an x-axis) and a second axis associated with detector response values (i.e. a y-axis);

b) selecting first and second time points defining a time span wherein peaks due to the homoduplex and heteroduplex molecules are located within the span;

c) for each profile and within the time span, adjusting the baseline by applying a slope factor to each detector response value. The factor is derived from a line connecting the detector response values at the first and second time points, such that all of the profiles share a common baseline;

d) for each profile and within the time span, normalizing the heights of the peaks to a pre-selected scale (such as 0-1) based on the height of the highest peak; and,

e) shifting the profiles along the first axis such that all of the profiles intersect at a pre-selected point on the last eluting peak of each profile within the span.

In one embodiment of the method, the pre-selected value is preferably zero, the pre-selected scale is from 0 to 1, the pre-selected point is a point on the last eluting edge of the last eluting peak, and in step (c) the second axis value at the first time point and the second axis value at the second time point are set to zero.

The elution profiles can include at least one reference profile obtained from a standard mixture resulting from the hybridization product of DNA having a known sequence and corresponding wild type DNA.

In another aspect, the invention provides a method for estimating the number of different single nucleotide polymorphisms in a plurality of same length DNA fragments. The method includes:

a) hybridizing each of the same length DNA fragments with corresponding wild type DNA to form homoduplex and heteroduplex molecules;

b) analyzing the hybridization product of each of the same length DNA fragments by Denaturing Matched Ion Polynucleotide Chromatography to obtain a plurality of elution profiles;

c) transforming the elution profiles by the method described hereinabove;

d) sorting the transformed profiles into a number of groups based on the shapes of the transformed profiles, wherein the number of single nucleotide polymorphisms is at least the same as the number of the groups.

In yet another aspect, the invention provides a method for detecting the presence of a previously unknown single nucleotide polymorphism in a test DNA fragment. The method preferably includes:

a) hybridizing the test DNA fragment with corresponding wild type DNA

b) analyzing the product of step (a) by Denaturing Matched Ion Polynucleotide Chromatography to obtain a test elution profile,

c) hybridizing standard DNA fragments with the wild type DNA,

d) analyzing the product of step (c) by Denaturing Matched Ion Polynucleotide Chromatography to obtain reference elution profiles,

e) obtaining a plurality of profiles by combining the test elution profiles and the reference elution profiles,

f) transforming the plurality of profiles by the method described herein,

g) sorting the plurality of profiles into groups based on the shapes of the plurality of elution profiles,

h) after the transforming, comparing the test elution profile with the groups.

A test DNA fragment is considered to contain a previously unknown mutation if the shape of the test elution profile does not match the shapes of the profiles in the groups.

The method can further include subjecting the test DNA fragment to a sequencing technique.

The method for transforming elution profiles can include applying one or more statistical criteria to the transformed profiles obtained after step (e) to determine whether or not to group the transformed profiles into a single group.

The statistical criteria can include:

a) within the time span, dividing the first axis into a series of adjacent and evenly-spaced time regions wherein boundary lines, perpendicular to the first axis, are defined between adjacent regions, and wherein the profiles intersect the boundary lines at intersecting detector response values,

b) for each boundary line

i) obtaining the mean of the intersecting detector response values, and comparing the mean to first a pre-selected value,

ii) obtaining the standard deviation of the mean of the intersecting detector response values, and comparing the standard deviation to a second pre-selected value,

iii) obtaining the range of the intersecting detector response values, and comparing the range to a third pre-selected value.

In still another aspect, the invention provides a computer implemented method for grouping a plurality of transformed chromatographic elution profiles

One embodiment of the method includes:

a) within the time span, dividing the first axis into a series of adjacent and evenly-spaced time regions wherein boundary lines, perpendicular to the first axis, are located between adjacent time regions, wherein the profiles intersect the boundary lines,

b) for each boundary line and between the highest intersecting profile and the lowest intersecting profile, dividing each boundary line into a plurality of equally spaced and adjacent segments,

c) for each boundary line, numbered 1 through i

i) determining the number of profiles intersecting each of the segments,

ii) determining the segment having the highest number of intersecting profiles (highest frequency segment) and determining the nearest segment having zero intersecting profiles (zero frequency segment),

iii) for each boundary line, assigning a numerical grouping factor of nⁱ to the profiles that have a second axis value greater than the segment having zero intersecting profiles and assigning a grouping factor of 1 to the remaining intersecting profiles, wherein n is an integer greater than 1,

d) for each profile, obtaining a total value comprising the sum of all the grouping factors assigned to the profile,

e) grouping together those profiles having the same total value.

In one embodiment the value of n is 2.

Another embodiment of the method for grouping a plurality of transformed chromatographic elution profiles includes:

a) placing one or more markers, numbered 1 through i, each marker placed at a position where the transformed elution profiles show apparently clustered detector response values,

b) obtaining the first axis value and second axis value for each marker, each marker located on a boundary line perpendicular to the first axis,

c) for each marker, and along its associated boundary line, assigning a numerical grouping factor of nⁱ to the profiles that have a second axis value greater than the second axis value of each marker, or otherwise assigning a grouping factor of 1 to the profiles, wherein n is an integer greater than 1,

d) for each profile, obtaining a total value comprising the sum of all the grouping factors assigned to the profile,

e) grouping together those profiles having the same total value.

In another aspect, the invention concerns a system for transforming chromatographic elution profiles The system includes: a computer having a processor and memory, wherein the computer receives a set of data corresponding to a plurality of chromatographic elution profiles, wherein each profile is obtained from the separation of a DNA mixture by Denaturing Matched Ion Polynucleotide Chromatography, wherein each DNA mixture comprises homoduplex and heteroduplex molecules obtained from the hybridization of a sample DNA and its corresponding wild type DNA The processor preferably:

a) overlays the profiles on a coordinate system comprising a first axis associated with time values and a second axis associated with detector response values,

b) selects first and second time points defining a time span wherein peaks due to said homoduplex and heteroduplex molecules are located within the time span,

c) for each profile and within the span, adjusts the baseline by applying a slope factor to each detector response value, the factor derived from a line connecting the detector response values at the first and second time points, such that all of the profiles have a common baseline. Preferably, the second axis value at the first time point and the second axis value at the second time point are set to zero for all of the profiles.

d) for each profile and within the span, normalizes the heights of the peaks to a pre-selected scale based on the height of the highest peak,

e) shifts of the profiles along the first axis such that all of the profiles intersect at a pre-selected point on the last eluting peak of each profile within the time span.

In preferred embodiments of the system, the pre-selected value is zero, the pre-selected scale is from 0 to 1, the pre-selected point comprises a point on the last eluting edge of said last eluting peak, and in step (c), the second axis value at the first time point and the second axis value at the second time point are set to zero. Also in preferred embodiments, the processor applies one or more statistical criteria to the transformed profiles obtained after step (e) to determine whether or not to group the transformed profiles into a single group.

In other embodiments of the system, the processor:

f) within the span, divides the first axis into a series of adjacent and evenly-spaced time regions wherein boundary lines, perpendicular to the first axis, are located between adjacent time regions,

g) divides each boundary line into a plurality of equal and adjacent segments,

h) for each boundary line, numbered 1 through i

i) determines the number of profiles intersecting each of the segments,

ii) determines the segment having the highest number of intersecting profiles and determines the nearest segment having zero intersecting profiles,

iii) for each boundary line, assigns a numerical grouping factor of nⁱ to the profiles that have a second axis value greater than the segment having zero intersecting profiles and assigns a grouping factor of 1 to the remaining intersecting profiles, wherein n is an integer greater than 1,

i) for each profile, obtains a total value comprising the sum of all the grouping factors assigned to the profile,

j) groups together those profiles having the same total value. Preferably, the value of n is 2.

In another embodiment of the system, the processor

f) receives instructions for placing one or more markers, numbered 1 through i, each marker placed at a position where the transformed elution profiles show apparently clustered detector response values,

g) obtains the first axis value and second axis value for each marker, each marker located on a boundary line perpendicular to the first axis,

h) for each marker, and along its associated boundary line, assigns a numerical grouping factor of nⁱ to the profiles that have a second axis value greater than the second axis value of the marker, or otherwise assigns a grouping factor of 1 to the profiles, wherein n is an integer greater than 1,

i) for each profile, obtains a total value comprising the sum of all the grouping factors assigned to the profile,

j) groups together those profiles having the same total value.

In another aspect, the invention relates to a computer readable medium for storing computer readable instructions, the instructions being capable of programming a computer to perform a method. The method performed by the computer includes a method for transforming a plurality of chromatographic elution profiles as indicated herein. The method performed by the computer also preferably includes applying one or more statistical criteria to the transformed profiles obtained after step (e) to determine whether or not to group the transformed profiles into a single group.

In another aspect, the invention provides a computer readable medium for storing computer readable instructions, the instructions being capable of programming a computer to perform a method. The method includes the method for transforming such as described herein and further includes a method for grouping such as by one of the methods described herein.

In other aspects, the invention provides a plurality of transformed elution profiles and a plurality of elution profiles grouped by the methods described herein.

### BRIEF DESCRIPTION OF THE DRAWINGS

FIG. 1 is a schematic block diagram of an embodiment of a generic computer system useful for implementing the present invention.

FIG. 2 shows a schematic representation of a hybridization to form homoduplex and heteroduplex DNA molecules and the DMIPC elution profiles of the DNA molecules.

FIG. 3 shows the DMIPC elution profile for two different mutations from a single gene.

FIG. 4 illustrates the relationship between hypothetical polymorphisms in a DNA fragment and their DMIPC elution profile.

FIG. 5 illustrates a set of raw data showing a plurality of chromatographic profiles.

FIG. 6 is a schematic flow chart of a computer program that implements the transformation of chromatographic profiles.

FIG. 7 illustrates an overlay of the profiles from FIG. 5 and the selection of a time span including the hybridized DNA molecules.

FIG. 8 shows a magnified view of the overlayed profiles from FIG. 7 within the selected time span.

FIG. 9 shows the profiles from FIG. 8 after implementation of a transformation procedure.

FIG. 10 is a schematic flow chart of an embodiment of a computer program that implements the determination of whether or not to group chromatographic profiles in a single group.

FIG. 11 is a schematic flow chart of an embodiment of a computer program that implements the automatic grouping of transformed chromatographic profiles.

FIG. 12 is a schematic flow chart of an embodiment of a computer program that implements the automatic grouping of transformed chromatographic profiles.

FIG. 13 illustrates boundary lines used in implementing a computer program for grouping a set of transformed profiles.

FIG. 14 is a schematic flow chart of a computer program that implements the semi-automatic grouping of chromatographic profiles.

FIG. 15 is an example of the placement of markers on a set of transformed profiles during implementation of the semi-automatic computer program.

FIG. 16 is an example of the placement of markers on a set of transformed profiles during implementation of the semi-automatic computer program.

FIG. 17 illustrates a user interface showing the chromatographic profiles of a first group from the profiles shown in FIG. 9.

FIG. 18 illustrates a user interface showing the chromatographic profiles of a second group from the profiles shown in FIG. 9.

FIG. 19 illustrates a user interface showing the chromatographic profiles of a third group from the profiles shown in FIG. 9.

FIG. 20 illustrates a user interface showing the location of grouped samples on a 96-well plate.

FIG. 21 illustrates overlayed profiles within a selected time span.

FIG. 22 shows the profiles from FIG. 21 after transformation procedure.

FIG. 23 illustrates a first group of chromatographic profiles from the profiles shown in FIG. 22.

FIG. 24 illustrates a second group of chromatographic profiles from the profiles shown in FIG. 22.

### DETAILED DESCRIPTION OF THE INVENTION

In a general aspect, the present invention concerns methods and devices for extracting information from DMIPC elution profiles. The instant invention concerns methods and devices for analyzing chromatograms obtained from the DMIPC analysis of samples containing hybridized DNA fragments; for transforming all of the chromatograms obtained from the analysis of a plurality of samples so that they can be viewed and analyzed in a standardized format; for grouping the adjusted profiles based on their shape or pattern. The invention can be used to determine the number and identity of SNPs in the samples by considering characteristics of the grouped profiles. The invention includes methods and devices for facilitating the comparison of DMIPC elution profiles so that they can be more readily interpreted; for grouping the profiles based on their shapes; and for determining whether a plurality of profiles represents more than one group of profiles.

The term “chromatographic elution profile” as used herein is defined to include the data generated by the MIPC method when this method is used to separate double stranded DNA fragments. The chromatographic profile can be in the form of a visual display, a printed representation of the data or the original data stream.

A “homoduplex” is defined herein to include a double stranded DNA fragment wherein the bases in each strand are complimentary relative to their counterpart bases in the other strand.

A “heteroduplex” is defined herein to include a double stranded DNA fragment wherein at least one base in each strand is not complimentary to at least one counterpart base in the other strand. Since at least one base pair in a heteroduplex is not complimentary, it takes less energy to separate the bases at that site compared to its fully complimentary base pair analog in a homoduplex. This results in the lower melting temperature at the site of a mismatched base of a heteroduplex compared to a homoduplex.

The term “hybridization” refers to a process of heating and cooling a dsDNA sample, e.g., heating to 95° C. followed by slow cooling. The heating process causes the DNA strands to denature. Upon cooling, the strands re-combine, or anneal, into duplexes in a statistical fashion. If the sample contains a mixture of wild type and mutant DNA, then hybridization will form a mixture of hetero- and homoduplexes. Hybridization can be effected by heating the combined strands to about 90° C., then slowly cooling the reaction to ambient temperature over about 45 to 60 minutes. During hybridization, the duplex strands in the sample denature, i.e., separate to form single strands. Upon cooling, the strands recombine. If a mutant strand was present in the sample having at least one base pair difference in sequence than wild type, the single strands will recombine to form a mixture of homoduplexes and heteroduplexes.

The term “Matched Ion Polynucleotide Chromatography” (MIPC) as used herein is defined as a process for separating single and double stranded polynucleotides using non-polar separation media, wherein the process uses a counterion agent, and an organic solvent to release the polynucleotides from the separation media. MIPC separations can be completed in less than 10 minutes, and frequently in less than 5 minutes. MIPC systems (WAVE® DNA Fragment Analysis System, Transgenomic, Inc. San Jose, Calif.) are equipped with computer controlled ovens which enclose the columns. Mutation detection at the temperature required for partial denaturation (melting) of the DNA at the site of mutation can therefore be easily performed. The system used for MIPC separations is rugged and provides reproducible results. It is computer controlled and the entire analysis of multiple samples can be automated. The system offers automated sample injection, data collection, choice of predetermined eluting solvent composition based on the size of the fragments to be separated, and column temperature selection based on the base pair sequence of the fragments being analyzed. The separated mixture components can be displayed either in a gel format as a linear array of bands or as an array of peaks. The display can be stored in a computer storage device. The display can be expanded and the detection threshold can be adjusted to optimize the product profile display. The reaction profile can be displayed in real time or retrieved from the storage device for display at a later time. A mutation separation profile, a genotyping profile, or any other chromatographic separation profile display can be viewed on a video display screen or as hard copy printed by a printer.

Depending on the conditions, MIPC separates double stranded polynucleotides by size or by base pair sequence and is therefore a preferred separation technology for detecting the presence of particular fragments of DNA of interest. A separation system for mutation detection having the convenience, automation, sensitivity, and range of capabilities of MIPC has not been previously described.

When mixtures of DNA fragments are applied to an MIPC column, they are separated by size, the smaller fragments eluting from the column first. However, when MIPC is performed at an elevated temperature which is sufficient to denature that portion of a DNA fragment domain which contains a heteromutant site, then heteroduplexes separate from homoduplexes. MIPC, when performed at a temperature which is sufficient to partially denature a heteroduplex, is referred to as “Denaturing Matched Ion Polynucleotide Chromatography” (DMIPC). DMIPC is also referred to in the art as DHPLC.

The term “heteromutant” is defined herein to include a DNA fragment containing a polymorphism or non-complimentary base pair.

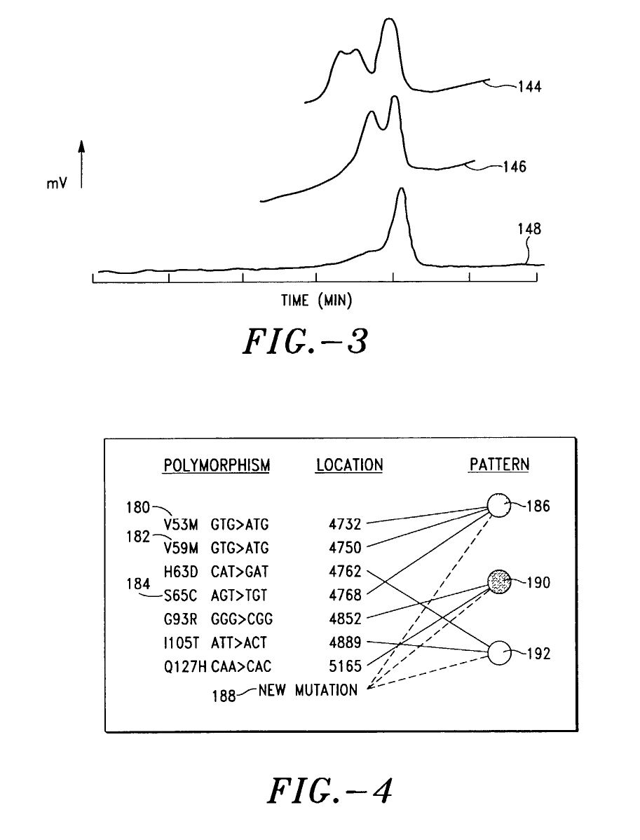

The term “mutation separation profile” is defined herein to include a DMIPC separation chromatogram which shows the separation of heteroduplexes from homoduplexes. Such separation profiles are characteristic of samples which contain mutations or polymorphisms and have been hybridized prior to being separated by DMIPC. The DMIPC separation chromatograms shown in FIG. 2 exemplifies mutation separation profiles as defined herein.

A reliable way to detect mutations is by hybridization of the putative mutant strand in a sample with the wild type strand (Lerman, et al., Meth. Enzymol., 155:482 (1987)). If a mutant strand is present, then two homoduplexes and two heteroduplexes will be formed as a result of the hybridization process. Hence separation of heteroduplexes from homoduplexes provides a direct method of confirming the presence or absence of mutant DNA segments in a sample. The temperature dependent separation of a 209 base pair mixture of homoduplexes and heteroduplexes by DMIPC is shown in FIG. 2. Prior to elution, a standard mixture, containing a mixture of homoduplex mutant and homoduplex wild type species, was hybridized as shown in the scheme 140. The hybridization process created two homoduplexes and two heteroduplexes. The standard mixture is available from Transgenomic, Inc., San Jose, Calif. (WAVE Optimized™ UV 209 bp Mutation Standard); the mutation is described by Seielstad et al., Hum. Mol. Genet. 3:2159 (1994). As shown in the elution profile 142, this mixture was separated using DMIPC. The two lower retention time peaks represent the two heteroduplexes and the two higher retention time peaks representing the two homoduplexes. The two homoduplexes separate because the A-T base pair denatures at a lower temperature than the C-G base pair. Without wishing to be bound by theory, the results are consistent with a greater degree of denaturation in one duplex and/or a difference in the polarity of one partially denatured heteroduplex compared to the other, resulting in a difference in retention time on the MIPC column.

In some mutation analyses, only two peaks or a partially resolved peak(s) are observed in DMIPC analysis. The two homoduplex peaks may appear as one peak or a partially resolved peak and the two heteroduplex peaks may appear as one peak or a partially resolved peak. In some cases, only a broadening of the initial peak is observed under partially denaturing conditions.

If a sample contained homozygous DNA fragments of the same length, then hybridization and analysis by DMIPC would only produce a single peak at any temperature since no heteroduplexes could be formed. In the operation of the DMIPC method, the determination of a mutation can be made by hybridizing the homozygous sample with the known wild type fragment and performing a DMIPC analysis at a partially denaturing temperature. If the sample contained only wild type fragments then a single peak would be seen in the DMIPC analysis since no heteroduplexes could be formed. If the sample contained homozygous mutant fragments, then analysis by DMIPC would indicate the separation of homoduplexes and heteroduplexes.

In another example of DMIPC analysis, FIG. 3 shows the separation of PCR amplified and hybridized fragments from a region of the Connexin-26 gene. The elution profiles from a first mutation 144, a second mutation 146, and wild type 148, are shown.

In the instant invention, the terms “transform,” “transforming” and “transformation” are defined herein to include computer implemented adjustment of signal data (usually represented along the y-axis) and time data (usually represented along the x-axis) of chromatographic traces to values acceptable for use in the grouping method described herein. The transformation process preferably includes steps for adjusting the baseline, the heights of the peaks, and the position of the profiles along the x-axis, as indicated hereinbelow.

Embodiments of the present invention also relate to an apparatus for performing these operations. This apparatus may be specially constructed for the required purposes, or it may be a general purpose computer selectively activated or reconfigured by a computer program and/or data structure stored in the computer. The processes presented herein are not inherently related to any particular computer or other apparatus. In particular, various general purpose machines may be used with programs written in accordance with the teachings herein, or it may be more convenient to construct a more specialized apparatus to perform the required method steps. The required structure for a variety of these machines will appear from the description given below.

In addition, embodiments of the present invention further relate to computer readable media or computer program products that include program instructions and/or data (including data structures) for performing various computer-implemented operations. The media and program instructions may be those specially designed and constructed for the purposes of the present invention, or they may be of the kind well known and available to those having skill in the computer software arts. Examples of computer-readable media include, but are not limited to, magnetic media such as hard disks, floppy disks, and magnetic tape; optical media such as CD-ROM disks; magneto-optical media such as floptical disks; and hardware devices that are specially configured to store and perform program instructions, such as read-only memory devices (ROM) and random access memory (RAM). Examples of program instructions include both machine code, such as produced by a compiler, and files containing higher level code that may be executed by the computer using an interpreter.

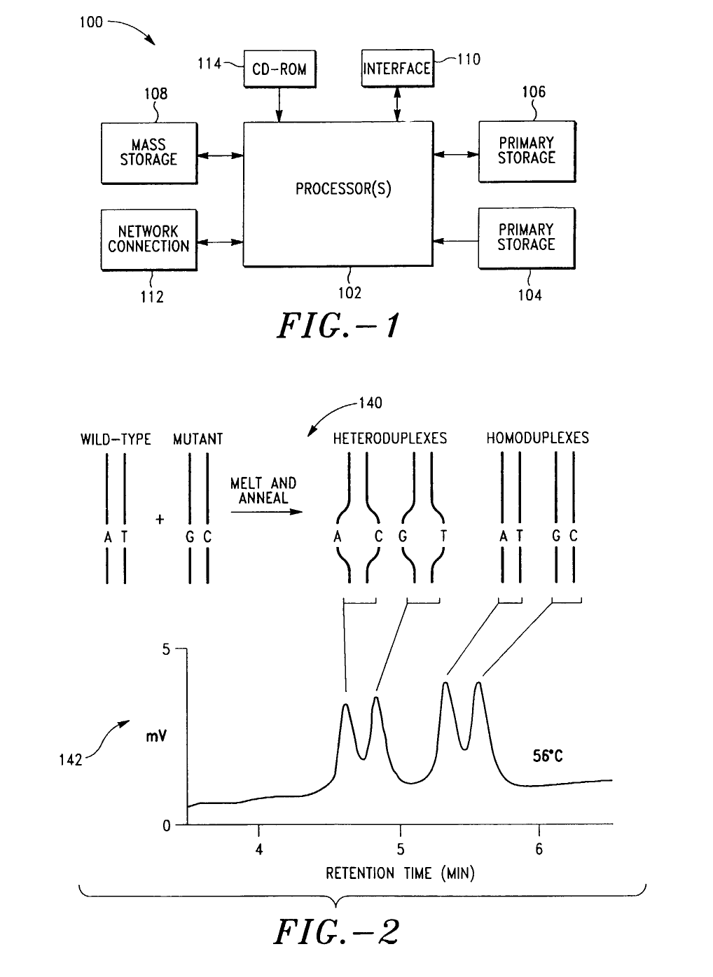

FIG. 1 illustrates a typical computer system in accordance with an embodiment of the present invention. The computer system 100 includes any number of processors 102 (also referred to as central processing units, or CPUs) that are coupled to storage devices including primary storage 106 (typically a random access memory, or RAM), primary storage 104 (typically a read only memory, or ROM). As is well known in the art, primary storage 104 acts to transfer data and instructions uni-directionally to the CPU and primary storage 106 is used typically to transfer data and instructions in a bi-directional manner Both of these primary storage devices may include any suitable computer-readable media such as those described above. A mass storage device 108 is also coupled bi-directionally to CPU 102 and provides additional data storage capacity and may include any of the computer-readable media described above. Mass storage device 108 may be used to store programs, data and the like and is typically a secondary storage medium such as a hard disk that is slower than primary storage. It will be appreciated that the information retained within the mass storage device 108, may, in appropriate cases, be incorporated in standard fashion as part of primary storage 106 as virtual memory. A specific mass storage device such as a CD-ROM 114 may also pass data uni-directionally to the CPU.

CPU 102 is also coupled to an interface 110 that includes one or more input/output devices such as such as video monitors, track balls, mice, keyboards, microphones, touch-sensitive displays, transducer card readers, magnetic or paper tape readers, tablets, styluses, voice or handwriting recognizers, or other well-known input devices such as, of course, other computers. Finally, CPU 102 optionally may be coupled to a computer or telecommunications network using a network connection as shown generally at 112. With such a network connection, it is contemplated that the CPU might receive information from the network, or might output information to the network in the course of performing the above-described method steps. The above-described devices and materials will be familiar to those of skill in the computer hardware and software arts.

The hardware elements described above may implement the instructions of multiple software modules for performing the operations of this invention. For example, instructions for transforming chromatographic profiles or for grouping profiles may be stored on mass storage device 108 or 114 and executed on CPU 108 in conjunction with primary memory 106.

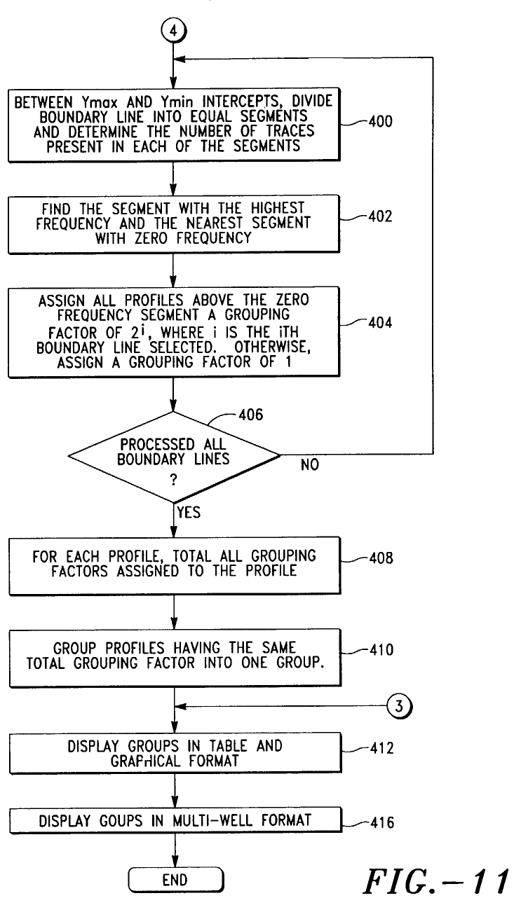

FIGS. 10-12 and 14 are process flow diagrams illustrating some of the important steps that may be employed in preferred embodiments of the present invention. At least some of these steps are implemented as software or machine operations on an appropriately configured computer system as described above. These operations are preferably performed sequentially, in a continuous fashion, by the software and/or appropriately configured machine. It is, of course, possible that various steps described here are performed by two or more separately operating pieces of software or appropriately configured machines.

As shown in FIG. 6, a process 300 begins at 302 and then in a step 304, the system receives data. After the appropriate data for the method has been input at step 304, the user next selected profiles to analyze at step 306. Next, at step 308, the system plots the selected profiles as overlayed profiles.

Those skilled in the relevant art can create source code (such as Visual Basic), microcode or program logic arrays or firmware based on the flowchart of FIGS. 6,10-12 & 14, and the detailed description provided herein. The routine 300 can be stored in the system memory 108 and/or non-volatile memory such as a magnetic disk.

The computer can be connected to a chromatography apparatus, or can be a stand alone computer separate from the chromatography apparatus. The methods and software of the invention are particularly useful in the “post-processing” of large amounts of chromatogrpahic data. When using a stand alone computer, the elution profile data can be loaded and stored into the computer memory through additional input means such as disk drives or tape drives, not shown in the drawings.

Embodiments of the present invention as described herein employ various process steps involving data stored in or transferred through computer systems. The manipulations performed in implementing this invention are often referred to in terms such as calculating, transforming, normalizing, adjusting, shifiting, or solving. Any such terms describing the operation of this invention are machine operations. Useful machines for performing the operations of embodiments of the present invention include general or special purpose digital computers or other similar devices. In all cases, there is a distinction between the method of operations in operating a computer and the method of computation itself. Embodiments of the present invention relate to method steps for operating a computer in processing electrical or other physical signals to generate other desired physical signals.

While routine 300 is presented as a defined sequence of process steps, the invention is not limited to software and systems that perform each and every one of these steps. For example, the invention is not limited to processes which perform steps in the exact order specified by the flow charts.

Referring now to FIG. 6, a data flow chart and control diagram is provided depicting the operation of the program. As shown by the flow chart of FIG. 6, after the method starts 302 the first step 304 in the process of the invention is to obtain the digital data files. These files will generally be the raw chromatogram data files produced by the control software provided with the chromatographic system. A preferred software is WAVEMAKER® software (Transgenomic, Inc., San Jose, Calif.), although other software could be used, such as available from Agilent (ChemStation), Shimadzu Class (VP data system), Waters (Millennium32 Software), Bio-Rad (Duo-Flow software), and Varian (ProStar Biochromatography HPLC System).

Once the data file or files to be evaluated have been obtained, the data from the files, is imported into the computer used to perform certain of the steps of the invention. The data may be imported into the computer via a diskette, CDrom or other media containing the data, or by direct link with another computer where the data is stored such as through a local area network, direct connection, ethernet, internet, etc.

An interface may be provided having several use actuated options such as the selection of one or more data files in a data batch to undergo analysis. Preferably, the computer is configured to permit importation of multiple data files by means of the import function while the use interface provides the user with the ability to select or deselect individual data files for inclusion in a batch file.

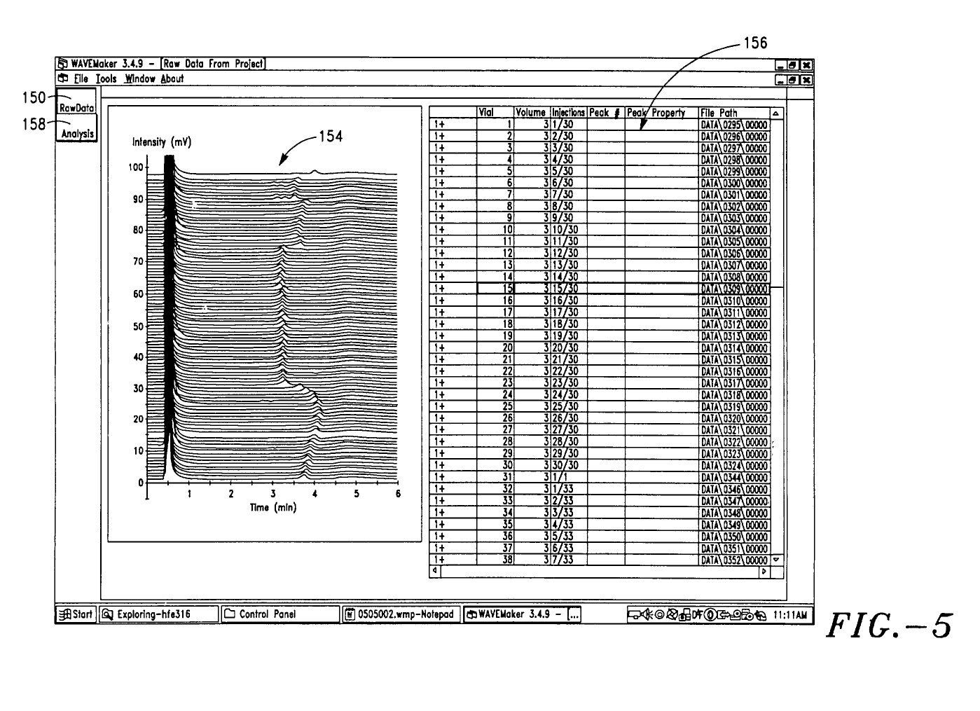

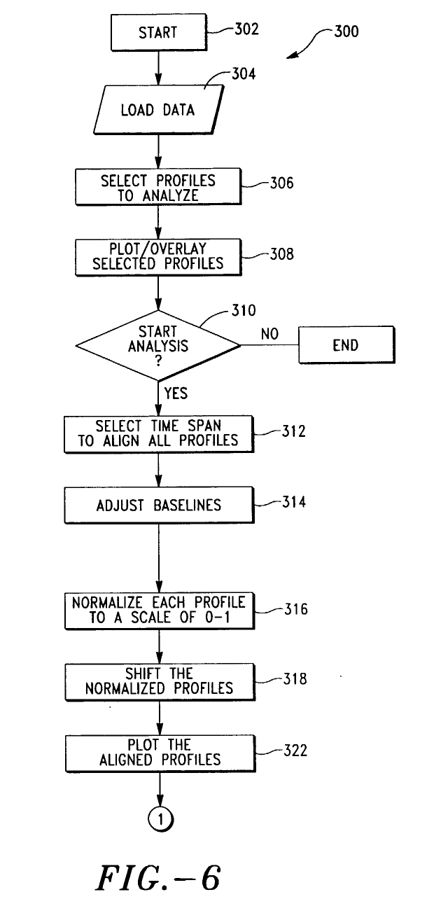

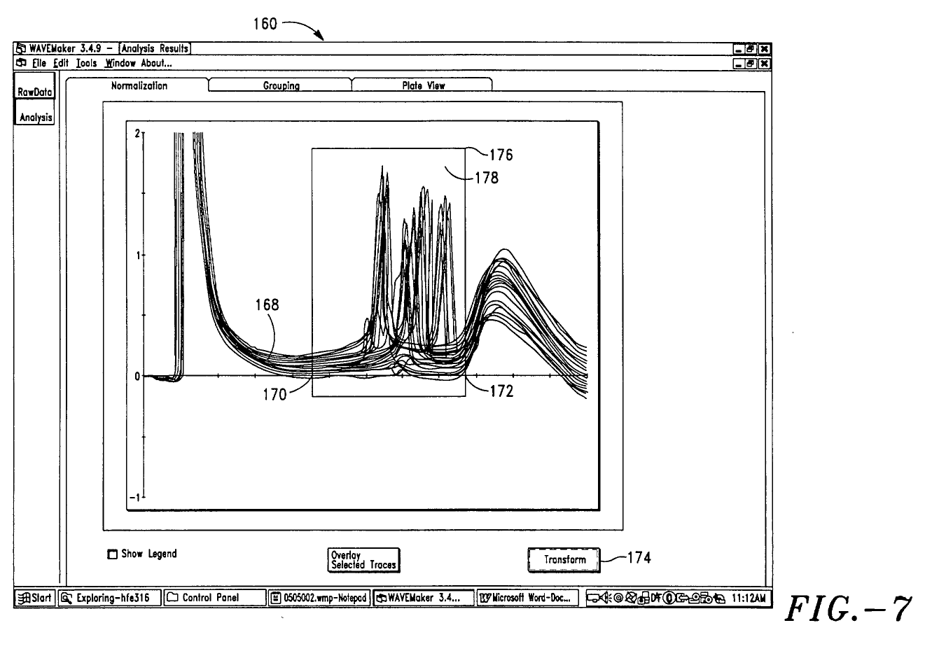

An example of a use screen is shown in FIG. 5 and can be activated by button 150. As depicted, pull down menus such as File, Tools, Window, and a Help menu can be made accessible to the user. Options available to the user under these means may be comparable to those of such widely known program such as Windows 3.1®, Windows 95®, Windows 2000®, Windows NT®, and Macintoch OS® interfaces. The interface preferably shows the chromatograms in a stacked format 154, and also a table format 156 showing the vial, volume of injection, peak number, peak property, and file path. At step 306, the use can selected the profiles to be analyzed. During the DMIPC elution, raw data consisting of digital data corresponding to the detector output (e.g., from UV or fluorescence detection) is stored along with associated time values. The time vs. signal data, or the image data, for the chromatograms can be stored on magnetic media, such as floppy disk, hard disk, tape, CD ROM or other media. A patent application identified by Ser. No. 09/039,061, and incorporated by reference herein, includes an example of computer systems for obtaining and displaying DMIPC chromatographic data.

The raw data is usually displayed as stacked chromatograms such as shown in FIG. 5 or as overlayed profiles such as shown in FIG. 7. It is impractical to identify groups of similar profiles using raw data as shown in FIGS. 5, 7, 8, and 21 due to slight run-to-run variations in baseline drift, detector signal noise, and retention time. These variations can be due to such factors as contamination of the separation column and changes in the composition of the mobile phase buffers (such as by evaporation of acetonitrile). The present invention is based in part on Applicants unexpected observation that when steps are taken transform the profiles obtained from DMIPC analysis, in order to correct for such variations, that groups of profiles can be observed. These groups can be used in determining the presence and identity of mutations in the DNA being analyzed.

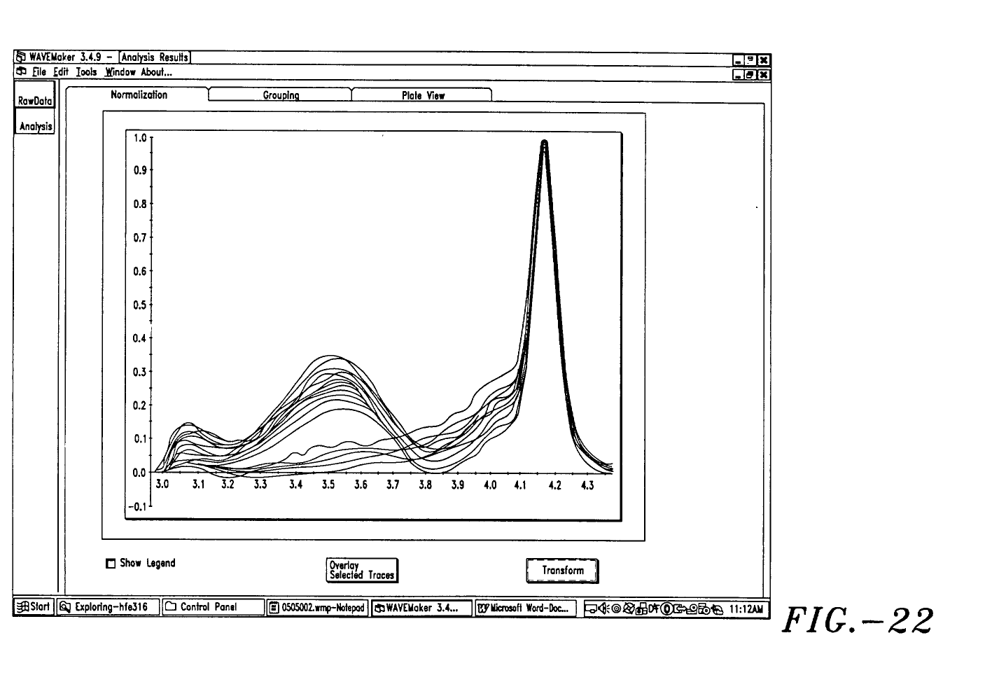

Another example of overlayed profiles is shown in FIG. 21 as described in Example 1. FIG. 21 shows 96 profiles from a complete multi-well plate containing both wild type and mutation samples. Slight base line and retention time variation are apparent which makes it difficult to see potentially differing patterns that may be present due to the wild type and the mutation containing samples.

Conventional software for presenting elution profiles as a series of stacked or overlapped traces is well known in the art. An example of a program for use in plotting multiple traces is Olectra Chart 6.0 (Apex Software Corp., Pittsburgh, Pa.) which can plot 96 traces in 12 seconds. It also has the ability to perform zooming and 3D plots.

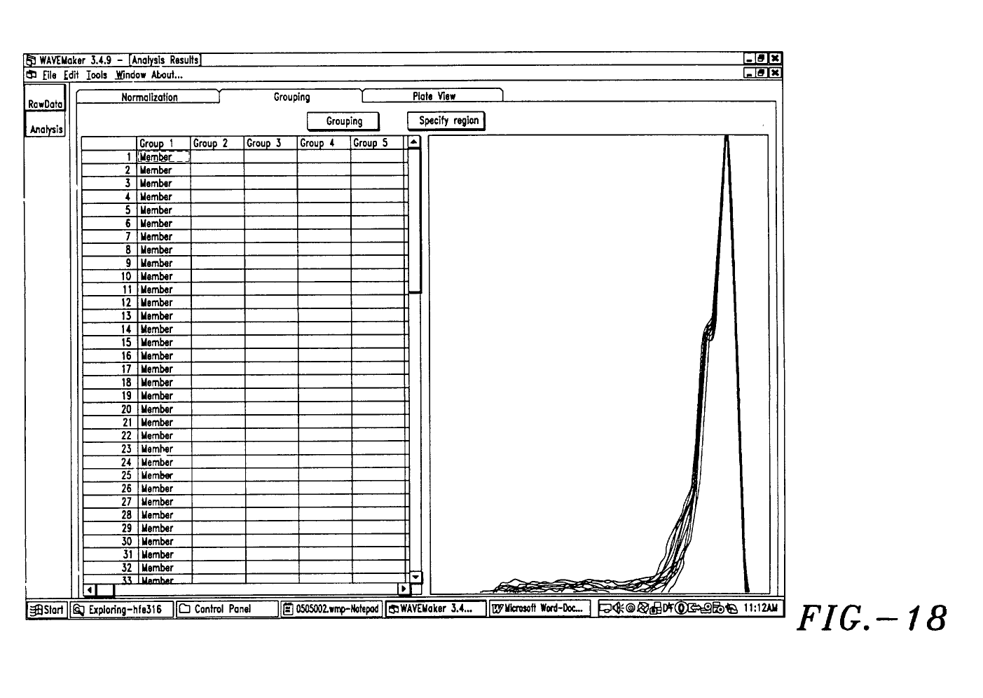

Once the files to be analyzed are selected, such as by highlighting, the user clicks the Analysis button 158. Once the Analysis button is pressed, an interactive Analysis Interface screen 160 pops up (FIG. 7). This interface provides the user with the option to view overlayed chromatograms 168 (step 308). The user can decide whether to proceed with the analysis or not at step 310. At step 312, the user selects a time span, including a first time 170 and a second time 172, for analysis. Once the time span is selected, the user can press the “Transform” button 174 to start the transformation process. Other interfaces include a Grouping interface 162 (FIG. 17) in which the user can view a table 164 showing all the members of each group, and a window 166 showing the profiles from a selected group.

In the instant invention, it will be understood that the signal vs. time data in the chromatograms can be presented on a variety of media, such as using a printer, or on a display device, such as a crt or flat panel screen. Images of the 96 well, or other multi well format, can also be displayed. Color coding can be used, for example to differentiate various chromatograms or locations of wells. The color coding can be used to highlight the grouping of chromatograms or to highlight groups of wells.

In general, the instant invention concerns methods and devices for analyzing chromatographic elution profiles obtained from the chromatographic analysis of DNA.

Examples of suitable chromatographic methods include MIPC and ion exchange chromatography either of which can be carried out under partially denaturing conditions. Anion exchange chromatography can be used for the separation of homoduplex and heteroduplex molecules as demonstrated in U.S. patent application Ser. Nos. 09/687,834 filed Jan. 11, 2000 and 09/756,070 filed Jan. 6, 2001. For purposes of clarity and not by way of limitation, MIPC and DMIPC are described herein.

In preparing a set of DNA fragments for analysis by DMIPC, it is assumed that all of the fragments have the same length since they are generated using the same set of PCR primers. It is further assumed that the fragments are eluted under essentially the same conditions of temperature and solvent gradient. The pattern or shape of the elution profile consists of peaks representing the detector response as various species elution during the separation process. The profile is determined by, for example, the number, height, width, symmetry and retention time of peaks. Other patterns can be observed, such as 3 or 2 peaks. The profile can also include poorly resolved shoulders. An example is shown in FIG. 3, showing a fragment from the connexin-26 gene (associated with sensori-neural deafness). There were two distinct patterns for two different mutations. The shape of the profile contains useful information about the nature of the sample. The pattern or shape of the resulting chromatogram will be influenced by the type and location of the mutation. Each SNP has a corresponding elution profile, or signature, at a given set of elution conditions of temperature and gradient. However, in using the present invention, Applicants have unexpectedly discovered that it is not true that each profile implies a unique SNP.

FIG. 4 schematically shows, in a hypothetical example, the relationship of a series of SNPs to their DMIPC elution pattern. In using the methods and devices of the instant invention as described herein, Applicants have surprisingly found that in some cases, different mutations can give the same elution profile. For example, the mutations shown at 180, 182, 184 all give the same pattern as shown at 186.

A DNA fragment having a new (previously unidentified) mutation, such as shown at 188, may give an elution profile that is indistinguishable from any of the existing (previously identified) profiles, such as shown at 186, 190 and 192. Another possibility is that the DNA fragment having a new mutation could yield an elution profile that is different from existing profiles.

A general aspect of the instant invention concerns the analysis of multiple chromatograms in order to group them by shape. Multiple, overlapping chromatograms are displayed on a display device. Chromatograms having matching profiles are grouped together. Applicants have surprisingly found that these groups can be used to detect the presence of mutations in the DNA samples being analyzed, and that the groups can also be used in the discovery of unknown mutations.

One aspect of the present invention concerns a method and device for transforming a plurality of DMIPC chromatographic profiles. This aspect of the invention generally provides for the adjustment of one, and preferably all three of the following profile parameters for each of the profiles: the baseline, the peak height, the retention time.

In one embodiment, the transforming process includes:

a) A step for selecting a time span which encompasses the homoduplex and heteroduplex peaks in the overlayed profiles.

b) A step for adjusting the baseline of all of the profiles. Preferably, the slopes of the baselines for all of the profiles are adjusted to be equal to each other. More preferably, each slope is adjusted to zero.

c) A step for normalizing the height of the profiles. Preferably, this includes, for each profile, normalizing all of the data points on the y-axis to a pre-selected scale based on the height of the highest peak. A preferred pre-selected scale is from 0 to 1.

d) A step for shifting the retention times of all of the profiles being analyzed so that all of the profiles overlap at a single pre-selected point.

More specifically, this aspect concerns a computer implemented method and device for transforming a plurality of chromatographic elution profiles wherein each profile is obtained from the separation of a DNA mixture by Denaturing Matched Ion Polynucleotide Chromatography, wherein each mixture comprises homoduplex and heteroduplex molecules obtained from the hybridization of a sample DNA and its corresponding wild type DNA. The method preferably includes the following steps:

a) Overlaying the profiles on a coordinate system comprising a first axis showing time values and a second axis showing detector response values.

b) Selecting first and second time points defining a time span wherein peaks due to the homoduplex and heteroduplex molecules are located within the span.

c) For each profile and within the span, adjusting the baseline by applying a slope factor to each detector response value. The factor is derived from a line connecting the detector response values at the first and second time points. After the baseline adjustment, the second axis value at the first time point and the second axis value at the second time point are set to a pre-selected value that is the same for all of the profiles. A preferred pre-selected value is zero at the first and second time points for all of the profiles.

d) For each profile and within the span, normalizing the heights of the peaks to a pre-selected scale based on the height of the highest peak. A preferred pre-selected scale is 0 to 1.

e) Shifting all of the profiles along the first axis such that all of the profiles intersect at a pre-selected point on the last eluting peak within the time span. A preferred pre-selected point is a point on the last eluting edge (i.e. the descending eluting edge) of the last eluting peak in the time span. A more preferred point is the point at half the peak height on the last eluting edge of the last eluting peak.

At step 312, first and second time points (e.g. as shown at 170 and at 172, respectively, in FIG. 7) defining a time span can be selected by the user by use of a pointing device, e.g., by mouse or touch pad. Preferably all of the homoduplex and heteroduplex molecules for all of the profiles are enclosed within the time span. In general, the initial time is selected to be after the retention time of the wash-through peak, but before the peaks due to dsDNA appear. Preferably, a relatively flat section is selected. The final time is selected after the appearance of the peaks due to the homoduplex fragments but prior to the broad peak that appears during the wash off phase of the elution (such a broad peak is shown at t=5 min in FIG. 7). Preferably, a relatively flat section is selected. The embodiment in FIG. 7 shows the use of a selection box 176 to select a rectangular area 178 enclosing the peaks of interest.

Referring to FIGS. 6 and 7, the invention preferably includes a step 314 for adjusting the baseline of all of the profiles. Preferably, the slopes of the baselines are adjusted to be equal. More preferably, each slope is adjusted to zero. In a preferred embodiment, for each profile, the y-values at the first and second time points are set to same value (i.e. to give a baseline slope of zero) while also proportionally adjusting all of the other points in the profile.

The profiles are preferably adjusted such that all of the profiles have a common baseline and all of the profiles intersect at the first and at the second time points. Preferably, the baseline slope is set to zero. In one embodiment, for each profile and within said span, the baseline is adjusted by applying a slope factor to each detector response value, the slope factor derived from a line connecting the detector response values at the first and second time points, such that all of the profiles have a common baseline and all of the profiles intersect at the first and second time points. In a more preferred embodiment, for each profile and within the time span, the baseline is adjusted by applying a slope factor to each detector response value, the factor is derived from a line connecting the detector response values at the first and second time points, such that all of the profiles have a common baseline and the second axis value at the first time point and the second axis value at the second time point are set to zero,

The following is a more detailed description of an embodiment of a method for adjusting the baseline of a profile:

For each profile, the y-axis values of each point are shifted either up or down so that the profile at the first time and at the second time intersect the x-axis at y=0. In a preferred embodiment, this step is performed for each profile by determining a slope factor, m, and an adjustment constant, c, which are applied to all of the points of the profile. In determining the slope factor for a profile, the y-axis values at the first time point and at the second time point are obtained. The slope factor is determined from these y-axis values. The value of m is calculated by determining the following ratio: m=[y(at the first time point)−y(at the second time point)]/(time range). The value of the adjustment constant, c, is obtained by solving the equation 0=m·x+c, where x equals the time value at the first time point. Along each profile, for each x value, a y′ value is calculated according to an adjustment equation: y′=y−(m·x+c), where y is the original y-axis value. The values for m or for c for a profile can be either positive or negative, depending on the shape of the profile before adjustment. Each profile is re-plotted along the x-axis using the y′ values.

Thus, in a preferred embodiment, the adjustment equation is thus applied so that at the first and at the second time points, all the profiles intersect at the y-axis value of 0. Also, in a preferred embodiment, all of the profiles have a common baseline after the adjustment.

The transformation procedure preferably includes, for each profile and within the time span, a subsequent step (step 316 in FIG. 6) for normalizing the heights of the peaks to a pre-selected scale based on the height of the highest peak. In a preferred embodiment, for each profile, all of the points are divided by the height of the highest peak in the time span. Thus, the highest peak after normalization has a value of 1. In other words, for each profile, each point is normalized to a scale of 0-1 by taking the ratio of the y-axis value of each point and the highest y-axis value (Ymax) for the profile within the time span. In order to facilitate viewing the traces on a display device, the highest value of the y-axis within the time span can be set to 1.0 in the display.

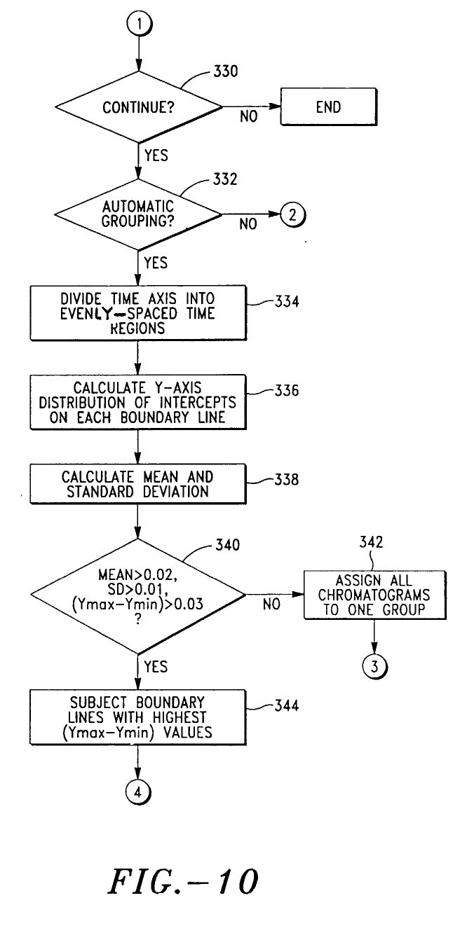

In step 318, the profiles are shifted along the x-axis so that all of the profiles overlap at a pre-selected single reference point. In one embodiment, the single reference point is a point on the last eluting peak. In a preferred embodiment, the single reference point is located on the last eluting edge of the last eluting peak in the time span. Most preferably, the point is located as having a y-axis value that is half the peak height of the last eluting peak in said time span. When the profiles are normalized to a scale of 0-1, this point corresponds to a y-axis value of 0.5 on the last eluting edge of the last eluting peak in the time span. An example of such a preferred point after shifting is shown at 320 (FIG. 9). The pre-selected reference point can be a point on the peak due to the elution of the wild type homoduplex fragment, and preferably is a point on the last eluting edge of this peak. In performing the shifting process, each profile is shifted (i.e. translocated) along the x-axis by either adding or subtracting a numerical factor to each x-axis value.

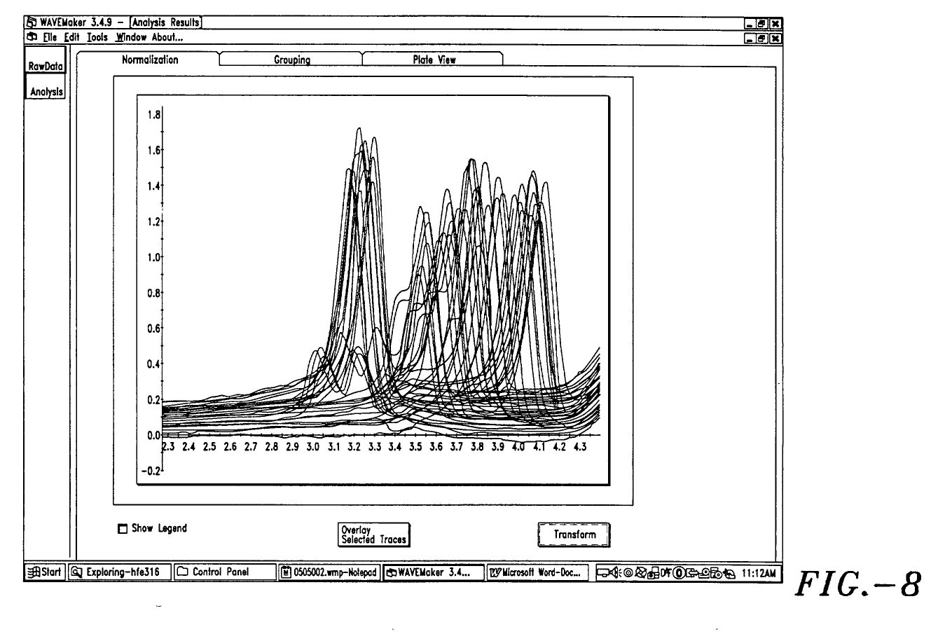

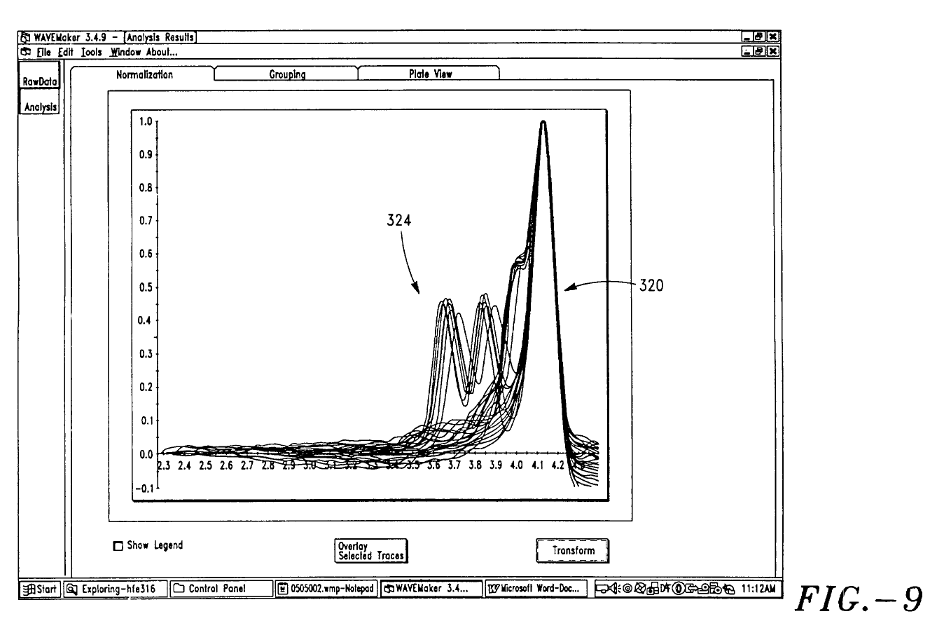

In step 322, the transformed profiles are plotted. Examples of transformed chromatographic profiles 324 and 326 (FIGS. 9 and 22, respectively) show a clearer presentation of the profiles thus facilitating their analysis, in contrast to the profiles prior to adjustment, FIGS. 8 and 21, respectively.

The data flow chart continues as shown in FIG. 10. At branch point 330 the user is prompted to proceed with automatic grouping. If the answer is “YES,” then the data flow continues to step 332.

In another aspect, the invention concerns applying one or more statistical criteria to the transformed profiles obtained after step 332 in FIG. 6 to determine whether or not to group said transformed profiles into a single group.

In one embodiment, the criteria include:

a) Within the selected time span, dividing the first axis of the transformed profiles into a series of adjacent and evenly-spaced time regions Boundary lines, perpendicular to the x-axis, and having a constant time value, are defined between adjacent regions. The profiles intersect the boundary lines at intersecting detector response values.

b) For each boundary line:

i) Obtain the mean of the intersecting detector response values, and compare the mean to first a pre-selected value.

ii) Obtain the standard deviation of the mean of the intersecting detector response values, and compare the standard deviation to a second pre-selected value.

iii) Obtain the range of the intersecting detector response values, and compare the range to a third pre-selected value.

This aspect of the invention is exemplified by the steps shown in FIG. 10. In step 334, the time axis within the selected time span is divided into a number of time regions, preferably regions of equal size. The number can be between about 3 and 1000 or higher, depending in part on the number of samples being analyzed. The time regions can comprise time intervals, such as seconds or fractions (e.g. tenths) of a second. An example of such a divided time axis is shown in FIG. 13 in which 5 regions (such as at 600 and 602) are shown. The number of regions will be selected based on factors such as the stringency of the test, the number of elution profiles being analyzed, the speed and memory capacity of the computer system. Boundary lines, such as at 604, 606, 608, 610, and 612 are defined between each of the time regions.

At step 336, the program determines the y-axis values where the profiles intersect each boundary line. At step 338, for each boundary line, the mean and standard deviation (SD) of these y-axis values is calculated.

The values used in the criteria shown at step 340 can be selected by the user. Examples of such criteria include:

A. Is the mean of the Y values greater than a pre-selected value?

B. Is the standard deviation greater than a pre-selected value?

C. Is the range (Ymax-Ymin) greater than a pre-selected value?

The values for the criteria shown at step 340 are preferred values, and were empirically found to be suitable when the number of boundary lines was 20 in step 334. The values can be selected by the user and are dependent on the stringency desired. The values selected can depend upon the number of boundary lines used, upon the number of samples, and upon the degree of confidence required for grouping the profiles into one group or into a plurality of different groups. In another embodiment, the following criteria were used in analyzing 96 samples, and where the number of boundary lines in step 334 was 12: Mean>0.01; SD>0.004; range>0.02.

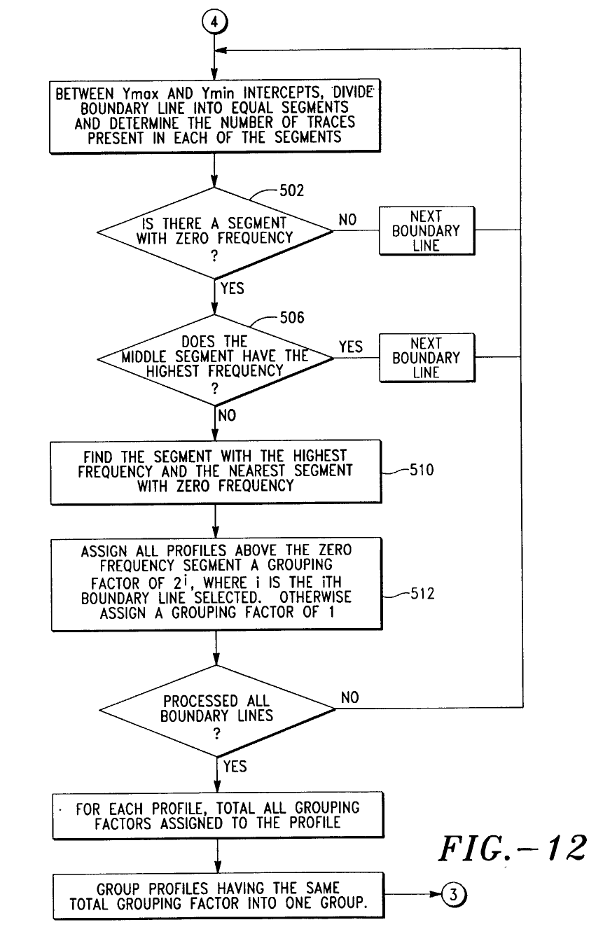

If none of the calculated values for the mean, SD, and range for any of the boundary lines exceed the values as stated in A, B or C, then the program flow continues along the arrow labeled “NO” to step 342, and all of the profiles are assigned, or classified, into one group. The profiles are then displayed in tabular and graphical format in step 412 and in multi-well format in step 414 (FIG. 11) as further described hereinbelow. owever, if for any boundary line, an affirmative answer to any of the criteria A, B or C is obtained, then it is concluded that there is a statistical difference in the profiles and that more than one group is present. The program proceeds along the arrow labeled “YES” to subsequent steps to further analyze the profiles in order to assign them into groups.

Selection of the values for the mean, SD and range can be based on empirical results. If the values for these criteria are set too high, the grouping will not be sensitive enough, and groups may be missed. However, if the values are set too low, grouping will be too sensitive, causing grouping into too many groups which may lead to false positives.

When selecting the values used for the SD and range (Ymax-Ymin) in step 340, a first set of chromatographic profiles which are known to represent identical samples can be used. These profiles can be generated, for example, by injecting many times from a single homogenous solution. The values of SD and of Ymax-Ymin can be varied; the values which cause this set of profiles to be grouped as two or more groups would be considered to be too stringent. The limiting values which lead to a single group are just stringent enough. In similar fashion, another set of profiles which are known nulls obtained from samples containing no DNA can be used. The limiting value for the mean which leads to a single group is just stringent enough.

If, based on the statistical criteria at step 340, it is concluded that there is more than one group of profiles, then the program continues at step 344.

In order to decrease computer processing time, a subset of the boundary lines can be selected for further processing. For example, in step 344, the boundary lines with the highest (Ymax-Ymin) values are selected by the program. This is a subset of the number of boundary lines selected in step 334. For example, the number of boundary lines in step 334 can be twenty and the number of boundary lines selected in step 344 can be five. Step 344 is optional, but preferred, since it decreases the time needed for computation.

As part of step 344, the boundary lines can be ranked from highest (Ymax-Ymin) to lowest (Ymax-Ymin). Each boundary line is assigned a number (1 through i), with boundary line 1 having the highest (Ymax-Ymin).

In a further aspect, the invention provides a computer implemented method and device for assigning a plurality of DMIPC elution profiles which have been transformed, as described herein, into groups. An embodiment of this aspect includes the following steps:

Within the time span, the x-axis is divided into a series of adjacent and evenly-spaced time regions wherein a boundary line, perpendicular to the x-axis and having constant time value, is located between adjacent time regions. An example of such a division is shown in FIG. 13 showing evenly-spaced regions, as exemplified by 600 and 602, and boundary lines 604, 606, 608, 610, and 612. The boundary lines are numbered 1 through i. In the hypothetical example shown in FIG. 13, the boundary lines 604, 606, 608, 610, and 612 are numbered 1, 2, 3, 4, and 5 respectively. Between the Ymax and Ymin intercept values, each boundary line is divided into a number of equally spaced and adjacent segments A, B, C, D, E, F, and G, such as shown at 620, 622, 624, 626, 628, 630, and 632, respectively. Each boundary line has an analogous series of segments. The elution profiles are labeled 650, 652, 654, and 656.

At step 400, for each boundary line, the number of profiles that intersect each segment is determined. In a preferred embodiment, for each boundary line, the Ymax value is included within the uppermost segment, and Ymin is included within the lowermost segment. If a profile intersects at the interface between two segments, it will be counted toward the lower segment. This gives a frequency map (TABLE I is based on FIG. 13).

TABLE I

Segment

| Boundary line | A | B | C | D | E | F | G |
|---|---|---|---|---|---|---|---|
| 604(i = 1) | 1 | 2 | 0 | 0 | 0 | 0 | 1 |
| 606(i = 2) | 1 | 0 | 0 | 0 | 0 | 2 | 1 |
| 608(i = 3) | 1 | 0 | 0 | 0 | 1 | 1 | 1 |
| 610(i = 4) | 1 | 0 | 0 | 0 | 0 | 1 | 2 |
| 612(i = 5) | 1 | 0 | 0 | 0 | 2 | 0 | 1 |

At step 402, for each boundary line, the segment having the highest number of intersecting profiles (highest frequency segment) is determined and the nearest segment having zero intersecting profiles (zero frequency segment) is determined. In one embodiment of this step, if the highest frequency segment is located at a y-axis value greater than a mid-point value of 0.5(Ymax-Ymin), then the zero frequency segment that is closest to and lower (i.e. having a lower y-value) than the highest frequency segment is used in subsequent steps. However, if the highest frequency segment is located at a y-axis value lower than the mid-point value, then the zero frequency segment that is closest to and higher (i.e. having a higher y-value) than the highest frequency segment is used in subsequent steps. If the mid-point value falls within the highest frequency segment, then the zero frequency segment that is closest to and lower (i.e. having a lower y-value) than the highest frequency segment is used in subsequent steps. These same determinations are made in relation to step 510 described hereinbelow.

At step 404, for each boundary line, a numerical grouping factor of nⁱ is assigned to the profiles that have a y-axis value greater than the y-axis values of the nearest zero frequency segment; a grouping factor of 1 is assigned to the remaining profiles. A value of n=2 is preferred because binary comparisons are readily handled by a computer, however other values of n could be used. n is preferably an integer greater than 1.

Grouping factors are assigned to all of the profiles until the decision at branch point 406 registers “YES”. This analysis is repeated at each boundary line from i=1-5. At step 408, for each profile, all of the assigned weighing factors are summed.

At step 410, the profiles are classified into separate groups, each group having the same total grouping factor. For each profile, a total value equal to the sum of all the grouping factors is assigned to that profile. Those profiles having the same total value are grouped together. An example of the assignment of grouping factors based on FIG. 13 and TABLE I is shown in the following TABLE II:

TABLE II

| Profile no. | Assigned Grouping Factor | | | | | Total |
|---|---|---|---|---|---|---|
| 650 | 2¹ | 2² | 2³ | 2⁴ | 2⁵ | 62 |
| 652 | 2¹ | 1 | 1 | 1 | 1 | 6 |
| 654 | 2¹ | 1 | 1 | 1 | 1 | 6 |
| 656 | 1 | 1 | 1 | 1 | 1 | 5 |

From TABLE II, it can be seen that profile 652 and profile 654 are grouped together into a single group since they have the same total of 6, whereas profile 650 and profile 656 are classified into separate groups since they have different total values.

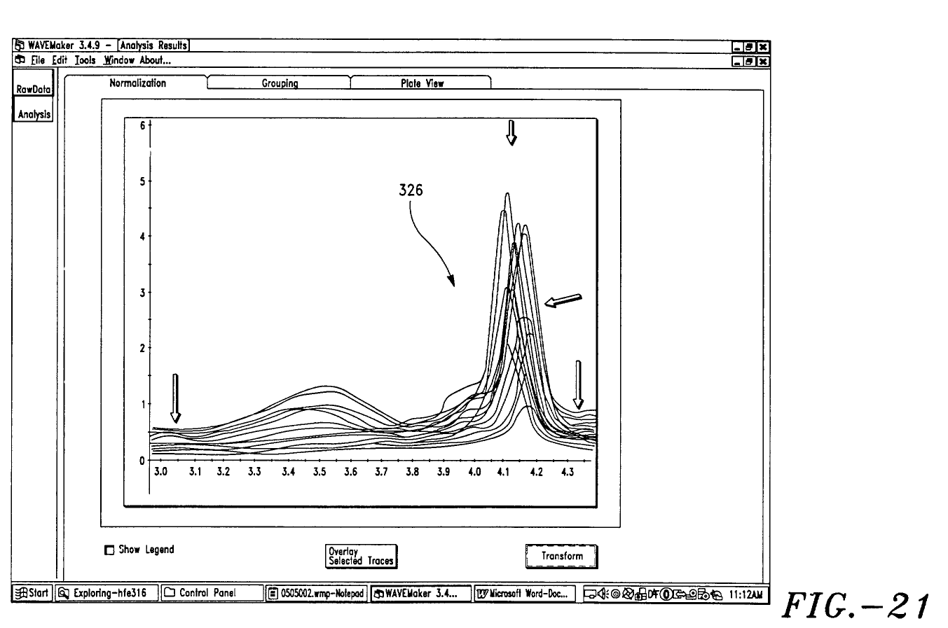

At steps 412 and 414, chromatograms are displayed numerically in a table, and as color coded traces in an x,y plot (e.g. as shown in FIG. 9). An example of a user interface screen showing a tabular display 166 and a graphical display 168 is shown in FIG. 17. The groups can also be displayed in a color coded multi-well format (such as at 169 in FIG. 20).

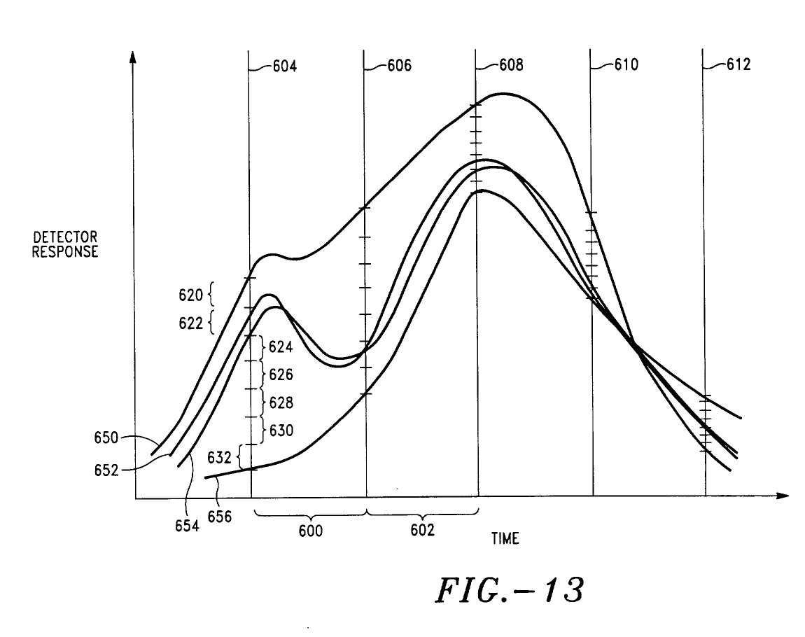

Another embodiment of the method is shown in FIG. 12. It will be seen that this embodiment includes two branch points, 502 and 506, that are inserted between steps 400 and 402 of the flow chart shown in FIG. 11. Branch point 502 queries whether the boundary line has zero frequency (i.e. zero count) of intersecting profiles. If “NO”, then this boundary line is disregarded in the subsequent grouping process. Without wishing to be bound by theory, the absence of zero frequency of intersecting profiles at step 502 is interpreted by as indicating that the profiles are dispersed randomly, and presumably, cannot be assigned into groups of distinct patterns of profiles. The dispersion is assumed to be due to noise, and thus disregarded for grouping.

Branch point 506 queries whether the segment with the highest frequency is the middle segment. If “YES”, then this boundary line is discarded in the grouping process. The profiles are evenly distributed about this segment, and are not useful in determining the grouping.

Steps 510 and 512 are the same as steps 402 and 404, respectively, as discussed hereinabove.

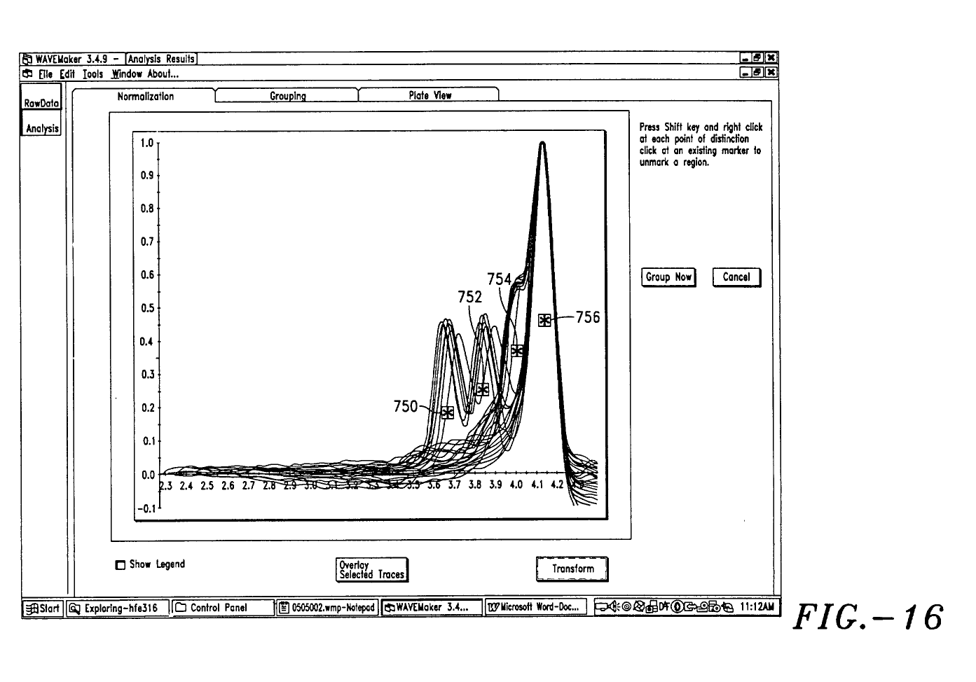

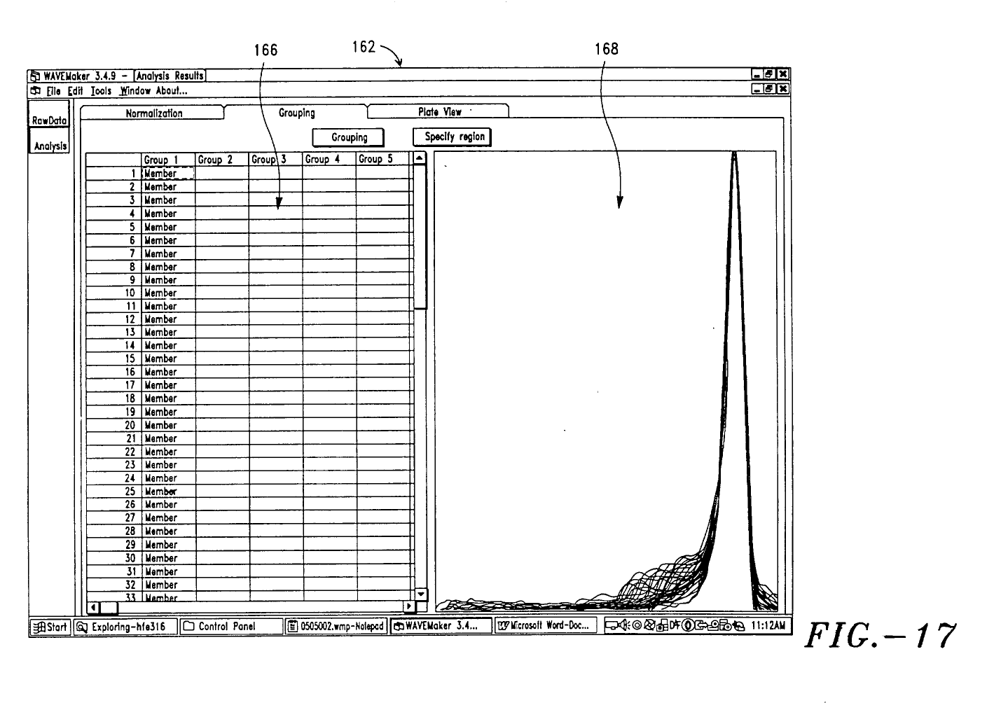

In another aspect, the invention concerns a method for grouping a plurality of transformed chromatographic elution profiles. An embodiment of this is a “semi-automatic” method for grouping which includes the steps indicated in FIG. 14. The semi-automatic (FIG. 14) method allows the user to visually identify possible groupings and can be used to confirm the results from the automatic method. At branch point 332, FIG. 10, the user is prompted to proceed with automatic grouping. If the answer is NO, then the data flow continues to the “semi-automated” procedure at step 700 (FIG. 14). At step 700, the user places markers, numbered 1 through i, on the transformed and overlapping profiles at points of distinction. The markers can be positioned by use of a pointing device, such as a mouse, at a graphical user interface. A “point of distinction” is defined herein to include a position where the transformed elution profiles are apparently clustered. The markers are preferably positioned between the apparent clusters. Thus, a region where all of the chromatograms densely overlap would preferably not be selected. Examples of such points of distinction are shown by the four markers located at 750, 752, 754, and 756 in FIG. 16.

At step 702, the x-axis and y-axis values for each marker is determined. Each marker located on a boundary line (such as shown at 736) parallel to the y-axis. Next, the chromatograms are sorted by assigning a grouping factor to them depending on their position in relation to the selected markers. At step 704, for each marker, and along its associated boundary line, a numerical grouping factor of nⁱ is assigned to the profiles that have a y-axis value greater than the y-axis value of the marker. Otherwise a grouping factor of 1 is assigned to the profiles on the boundary line. A value of n=2 is preferred, however other values of n could be used.

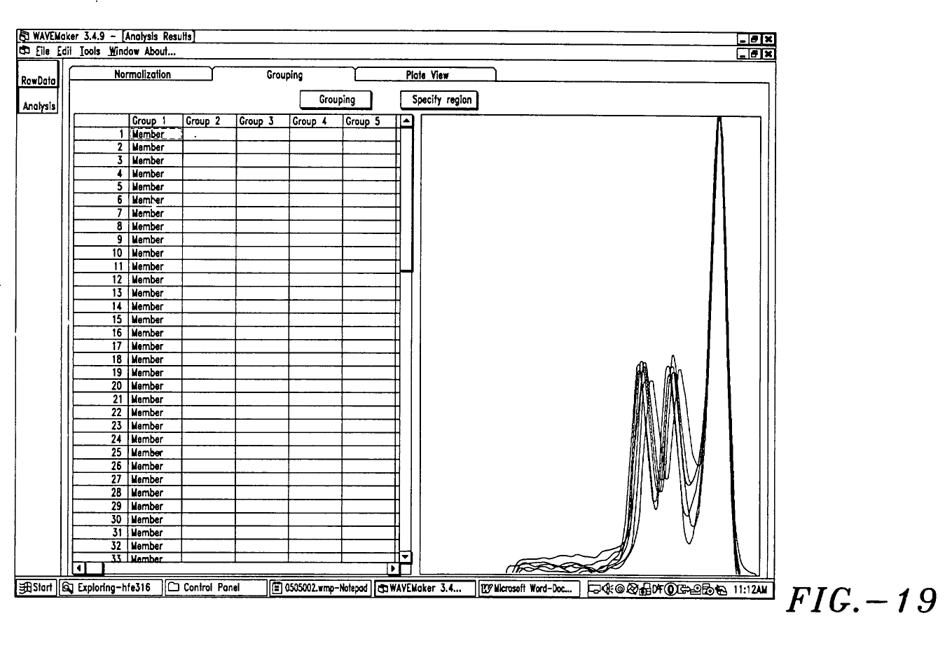

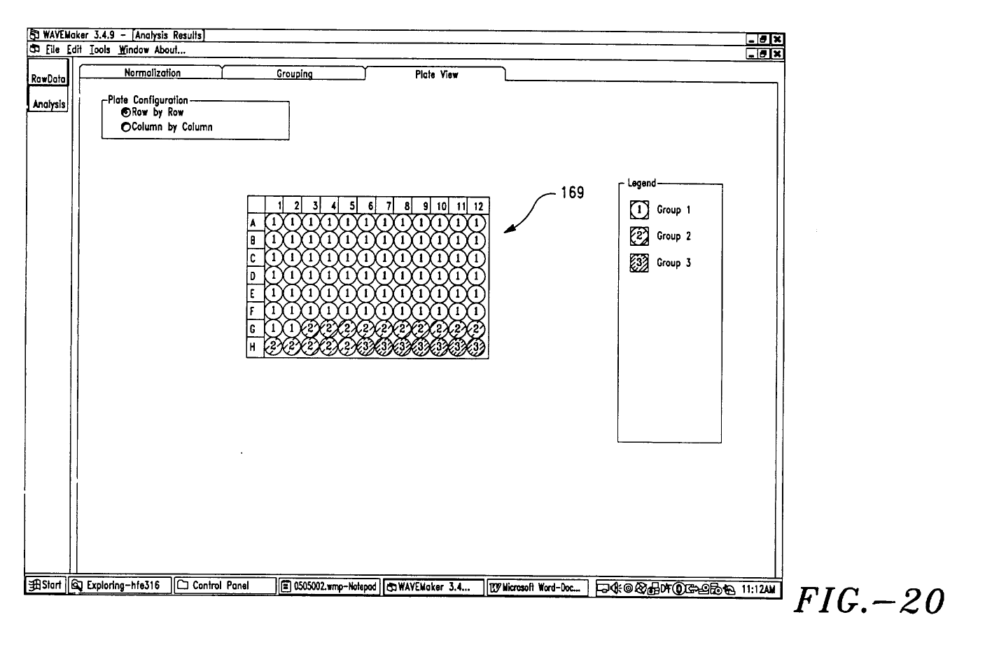

At step 706, for each profile, a total value equaling the sum of all the assigned grouping factors for that profile is obtained. At step 708, those profiles having the same total value are grouped together. Grouped chromatograms can then be displayed numerically in a table, and as color coded traces in an x,y plot. Examples of a user interface screen 162 showing a tabular display 166 and a graphical display 168 are shown in FIGS. 17-19. The groups can also be displayed in a color coded multi-well format (such as at 169 in FIG. 20).

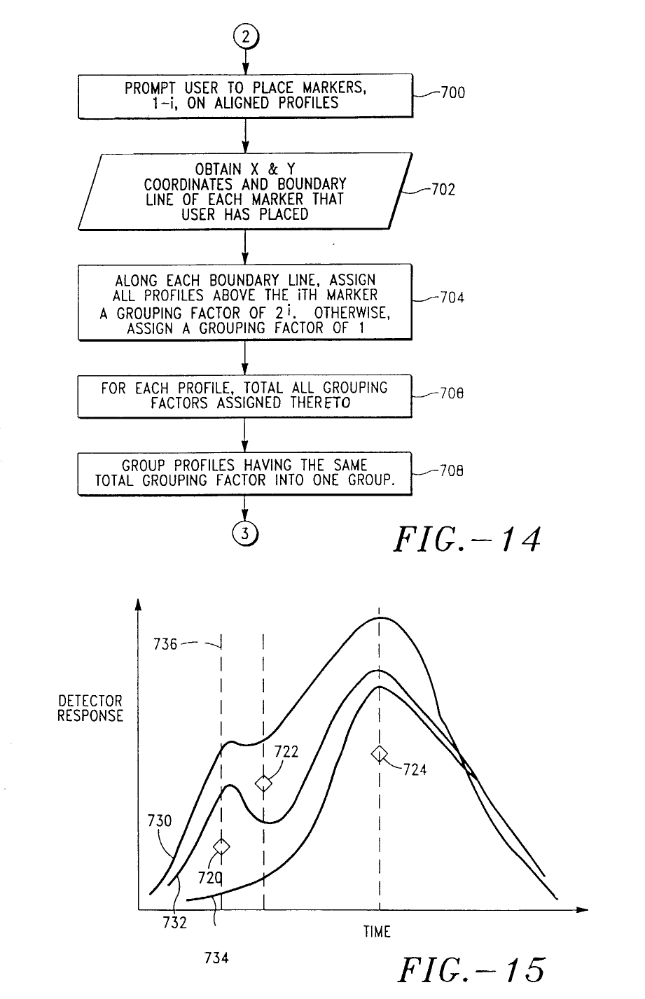

An example of the use of the semi-automatic method is illustrated in FIG. 15. For example, the user could have selected three markers as shown in FIG. 15 at 720, 722, and 724. The markers are assigned the values 2¹, 2², and 2³, respectively.

For example, for the first marker 720 (i=1), if there were two profiles above the marker, and one below, then each of the upper two profiles are assigned a value of 2¹ and the lower profile is assigned a value of 1. As another example, if there are three profiles above the second marker 724 (i=2), then each of these profiles is assigned a value of 2². As yet another example, if a third marker 722 (i=3) is positioned above two of the chromatograms, then the two lower profiles are assigned a value of 1 while the upper one is assigned a value of 2³.

At step 706, the assigned grouping factors are summed up for each profile. In the example shown in FIG. 15, this would yield the results shown in Table III

TABLE III

| Profile no. | Assigned Grouping Factor | | | Total |
|---|---|---|---|---|
| 730 | 2¹ | 2² | 2³ | 14 |
| 732 | 2¹ | 2² | 1 | 7 |
| 734 | 1 | 2² | 1 | 6 |

Thus profiles 730, 732 and 734 each have different total values and are grouped into three different group.

In the use of the method, a user need not proceed with the automatic or the semi-automatic grouping of the transformed profiles. For example, at branch point 330 in FIG. 10, a user can decide to end the program, view the plot of transformed profiles, and attempt to group the profiles by visual inspection.

In another aspect, the invention can be used for estimating the number of mutations present in a plurality of dsDNA samples, each DNA sample having either a wild type sequence or a single nucleotide polymorphism. One embodiment of this aspect of the invention is a method for determining the number of mutations (e.g. SNPs) in a plurality of samples, each sample comprising a fragment of double stranded DNA. This method can include a) hybridizing each sample with corresponding wild type double stranded DNA, wherein a mixture of homoduplex and heteroduplex molecules is formed if a sample contains a mutation; obtaining a plurality of sample chromatographic profiles, each sample profile from Denaturing Matched Ion Polynucleotide Chromatography analysis of one of said hybridized samples; superimposing and adjusting the sample profiles by the methods described herein; grouping the superimposed and adjusted sample profiles according to the pattern or shape of each sample profile using the methods described herein. Assuming that one of the groups is due to the presence of wild type dsDNA in some of the sample, then a lower limit for the number of different mutations in the dsDNA samples is n-1 where n is the number of different groups observed. The adjusted sample profiles can also be compared to a group of reference chromatographic profiles that have been similarly superimposed and adjusted, the reference profiles obtained from standard DNA fragments. Standard DNA fragments include mixtures of homoduplex mutant fragments and homoduplex wildtype fragments both of known sequence, such as the 209 bp standard described hereinabove, which are hybridized prior to DMIPC. The groups of reference chromatographic profiles can be generated contemporaneously with the sample profiles (such as analysis of standard DNA fragments in the same multi well plate), or can be obtained from previously analyzed standards. The sample profiles can be assigned to one or more of the groups from the reference profiles.

Further analysis can be used, for example, to confirm or reject the assignment of a sample chromatogram to a group obtained from reference chromatograms. Full sequencing can be performed using conventional sequencing methods such as the Maxam-Gilbert or the Sanger methods, and are described, for example, in Sambrook, J., Fritsch, E. F. and Maniatis, T. (1989) in Molecular Cloning: A Laboratory Manual, Second Edition, Cold Spring Harbour Laboratory Press, New York, or in Ausubel et al.(1995) in Current Protocols in Molecular Biology, John Wiley & Sons.

Conventional minisequencing methods can also be used and are more rapid and less costly than full sequencing. In minisequencing, a primer is designed that anneals adjacent to the variable nucleotide position in a template, and the primer is extended, in the presence of a polymerase, by at least one base in the presence of a terminator (e.g. a dideoxy nucleosidetriphosphate, ddNTP) and dNTPs. The incoming base or the primer can be chemically tagged in order to enhance detection. The length of the extended product indicates the identity of the base at the variable nucleotide position. The extension products can be separated by size using MIPC, gel electrophoresis or other conventional method. Minisequecing and primer extension methods are described in Hoogendoom et al. Human Genetics 104:89-93 (1999), and in U.S. Pat. Nos. 5,834,19; 5,484,701; and 5,273,638. Other methods can be used to verify the group assignment, such as enzymic or chemical cleavage, as described, for example, in U.S. Pat. Nos. 6,027,898; 6,187,539; and 5,869,245.

The instant invention provides methods and devices useful for identifying mutations which have not previously been identified. In addition, the invention can be used in instances where a particular mutation has been identified and an individual is screened to determine whether he or she possess the previously identified DNA mutation. The methods and devices of the instant invention are useful in detecting whether a set of samples contain new SNPs and in determining the identity of the SNPs. One embodiment of this use of the invention is exemplified by the following steps:

1. The DMIPC method is used to analyze a plurality of samples each of which contain test fragments having a previously uncharacterized SNP, or standard fragments having a known sequence.

2. The chromatographic profiles are grouped by shape as described herein.

3. Standard fragments that yield chromatographic profiles that do not match any of the shapes are eliminated from further consideration by this approach and can be subjected to an independent method such as a sequencing method.

4. A test fragment which yields a profile that matches one of the groups is subjected to a confirmatory test to determine whether or not the test sample has the same sequence at the site of variation as a known SNP in one of the standard fragments. This confirmatory test is preferably a method that does not require full sequencing. Examples of such methods include minisequencing and single base primer extension.

5. If a test sample yields a profile that does not match one of the groups, or if the test sample is not confirmed in step 4, then full sequencing is required to characterize the sequence.

### EXAMPLE 1

### Mutations in Haemochromatosis HFE Gene

This example demonstrates scanning for mutations in an amplicon from Homo Sapiens haemohromatosis HFE gene (Feder et al. Nature Genetics 13:399-408 (1996)). The sequence is found in GeneBank Accession no. CAB07442. The mutation is a G>A mutation at position 6722. This gives rise to a C282Y mutation in the expressed peptide.

In this example, the amplicon covers nucleotides 6614 to 6771 (158 bp product). The sequence of the amplicon is as follows:

(SEQ ID NO:1)

TGGAGCCAAGGAGTTCGAACCTAAAGACGTATTGCCCAATGGGGATGGGAC
CTACCAGGGCTGGATAACCTTGGCTGTACCCCCTGGGGAAGAGCAGAGATA
TACGTGCCAGGTGGAGACACCCAGGCCTGGATCAGCCCCTCATTGTGATCT
GGGGT

Sample DNA was amplified by conventional touchdown PCR methods (see U.S. Pat. Nos. 4,683,202 and 5,795,976) using a proof-reading polymerase and using the following primers.

Forward primer:

5′-TGGATGCCAAGGAGTTCGA (SEQ ID NO: 2)

Reverse primer:

5′-ACCCCAGATCACAATGAGGG (SEQ ID NO: 3)

After amplification, each sample was mixed with an equimolar amount of wild type dsDNA and hybridized. The mixture was separated using a WAVE® DNA Fragment Analysis System (Transgenomic, Inc., San Jose, Calif.) under the following conditions: Column: 50×4.6 mm ID containing alkylated poly(styrene-divinylbenzene) beads (DNASep®, Transgenomic); mobile phase 0.1 M TEAA (1 M concentrate available from Transgenomic) (Eluent A), pH 7.3; gradient: 50-53% 0.1 M TEAA and 25.0% acetonitrile (Eluent B).

FIG. 21 shows 96 elution profiles selected from DMIPC analysis of 96 amplified DNA samples. FIG. 22 shows the elution profiles of FIG. 21 within a selected time span after being subjected to the transformation method described herein.

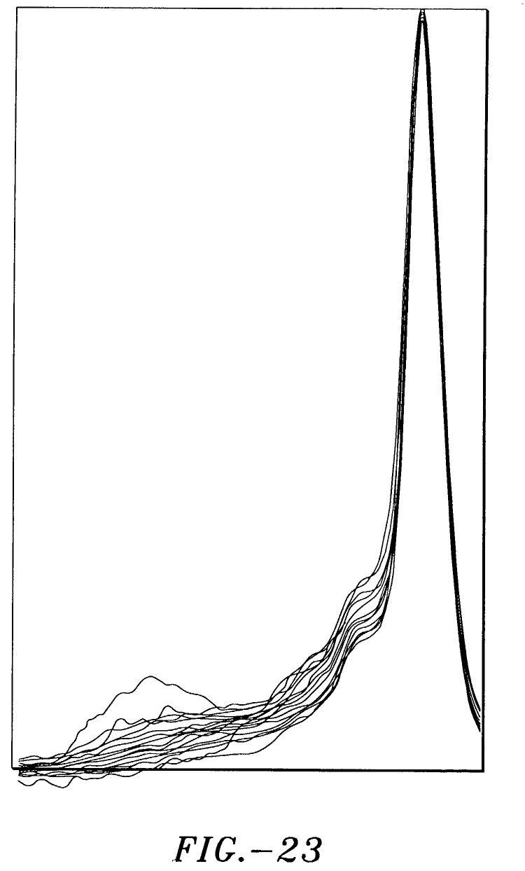

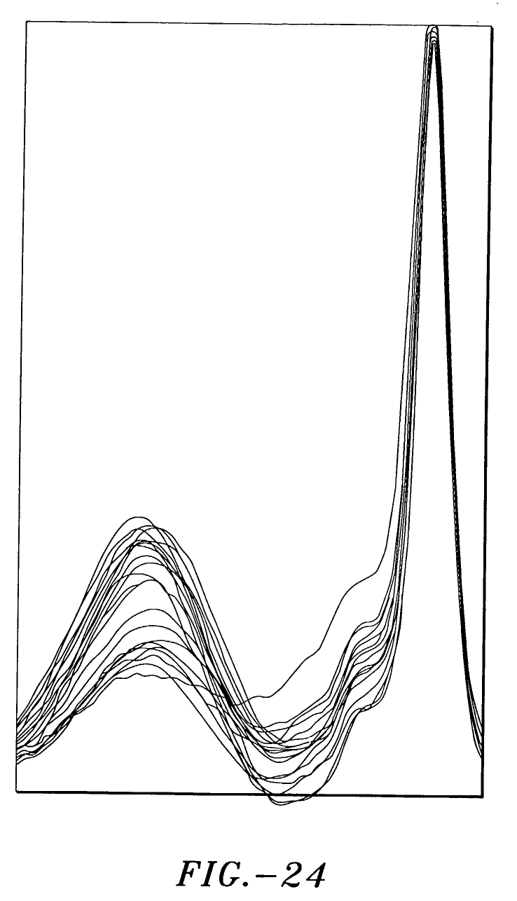

FIG. 23 shows a first group of elution profiles that was identified using the automated grouping method described herein. The DNA in the samples in this first group is the wild type DNA. FIG. 24 shows a second group that was identified using the automated grouping method. The DNA in this group was found to possess the G>A mutation. The grouping assignments obtained by the automated method were essentially the same as those obtained by a manual method, involving visual inspection and grouping, performed by four different individuals.

All references cited herein are hereby incorporated by reference in their entireties.

In the drawings and specification, there have been disclosed typical preferred embodiments of the invention and, although specific terms are employed, they are used in a generic and descriptive sense only and not for purposes of limitation, the scope of the invention being set forth in the following claims. Moreover, the terminology in the present description and claims relating to graphs, plotting lines, determining linear relationships, slopes, time regions, boundary lines and segments, etc. is intended to include the processing of data and variables internal to a processing unit (e.g., computer) containing memory and not limited to the physical acts of printing or plotting lines, curves, and graphs.

While the foregoing has presented specific embodiments of the present invention, it is to be understood that these embodiments have been presented by way of example only. It is expected that others will perceive and practice variations which, though differing from the foregoing, do not depart from the spirit and scope of the invention as described and claimed herein.

The invention claimed is:

1. A computer implemented method for transforming a plurality of chromatographic elution profiles, wherein each profile is obtained from the separation of a DNA mixture by Denaturing Matched Ion Polynucleotide Chromatography, wherein each DNA mixture comprises homoduplex and heteroduplex molecules obtained from the hybridization of a sample DNA and its corresponding wild type DNA, said method comprising:
a) overlaying said profiles on a coordinate system comprising a first axis associated with time values and a second axis associated with detector response values,
b) selecting first and second time points defining a time span wherein peaks due to said homoduplex and heteroduplex molecules are located within said span,
c) for each profile and within said span, adjusting the baseline by applying a slope factor to each detector response value, said factor derived from a line connecting the detector response values at said first and second time points, such that all of the profiles share a common baseline,
d) for each profile and within said span, normalizing the heights of the peaks to a pre-selected scale based on the height of the highest peak,
e) shifting the profiles along said first axis such that all of the profiles intersect at a pre-selected point on the last eluting peak of each profile within said span.

2. The method of claim 1 wherein said pre-selected value is zero, wherein said pre-selected scale is from 0 to 1, wherein said pre-selected point comprises a point on the last eluting edge of said last eluting peak, and wherein in step (c) the second axis value at the first time point and the second axis value at the second time point are set to zero.

3. The method of claim 1 wherein said profiles include at least one reference profile obtained from a standard mixture comprising the hybridization product of DNA having a known sequence and corresponding wild type DNA.

4. A method for estimating the number of different single nucleotide polymorphisms in a plurality of same length DNA fragments, said method comprising:
a) hybridizing each of said same length DNA fragments with corresponding wild type DNA to form homoduplex and heteroduplex molecules,
b) analyzing the hybridization product of each of said same length DNA fragments by Denaturing Matched Ion Polynucleotide Chromatography to obtain a plurality of elution profiles,
c) transforming said profiles by the method of claim 1,
d) sorting said transformed profiles into groups based on the shapes of said transformed profiles, wherein the number of single nucleotide polymorphisms is at least the same as the number of said groups.

5. A method for detecting the presence of a previously unknown single nucleotide polymorphism in a test DNA fragment, said method comprising:
a) hybridizing said test DNA fragment with corresponding wild type DNA
b) analyzing the product of step (a) by Denaturing Matched Ion Polynucleotide Chromatography to obtain a test elution profile,
c) hybridizing a standard mixture comprising DNA fragments of known sequence with said wild type DNA,
d) analyzing the product of step (c) by Denaturing Matched Ion Polynucleotide Chromatography to obtain reference elution profiles,
e) obtaining a plurality of profiles by combining said test elution profiles and said reference elution profiles,
f) transforming said plurality of profiles by the method of claim 1
g) sorting said plurality of profiles into groups based on the shapes of said plurality of elution profiles,
h) after said transforming, comparing said test elution profile with said groups, wherein said test DNA fragment is considered to contain a previously unknown mutation if the shape of said test elution profile does not match the shapes of the profiles in said groups.

6. The method of claim 5 further including subjecting said test DNA fragment to sequencing.

7. The method of claim 1 including applying one or more statistical criteria to the transformed profiles obtained after step (e) to determine whether or not to group said transformed profiles into a single group.

8. The method of claim 7 wherein said statistical criteria comprise:
a) within said span, dividing the first axis into a series of adjacent and evenly-spaced time regions wherein boundary lines, perpendicular to said first axis, are defined between adjacent regions, and wherein said profiles intersect said boundary lines at intersecting detector response values,
b) for each boundary line
i) obtaining the mean of the intersecting detector response values, and comparing said mean to a first pre-selected value,
ii) obtaining the standard deviation of the mean of the intersecting detector response values, and comparing said standard deviation to a second pre-selected value,
iii) obtaining the range of the intersecting detector response values, and comparing said range to a third pre-selected value.

9. A computer implemented method for grouping a plurality of transformed chromatographic elution profiles obtained by the method of claim 1, said method for grouping comprising:
a) within said span, dividing the first axis into a series of adjacent and evenly-spaced time regions wherein boundary lines, perpendicular to said first axis, are located between adjacent time regions, wherein said profiles intersect said boundary lines,
b) for each boundary line and between the highest intersecting profile and the lowest intersecting profile, dividing said each boundary line into a plurality of equally spaced and adjacent segments,
c) for each boundary line, numbered 1 through i
i) determining the number of profiles intersecting each of said segments,
ii) determining the segment having the highest number of intersecting profiles and determining the nearest segment having zero intersecting profiles,
iii) for each boundary line, assigning a numerical grouping factor of nⁱ to the profiles that have a second axis value greater than said segment having zero intersecting profiles and assigning a grouping factor of 1 to the remaining intersecting profiles, wherein n is an integer greater than 1,
d) for each profile, obtaining a total value comprising the sum of all the grouping factors assigned to said each profile,
e) grouping together those profiles having the same total value.

10. The method of claim 9 wherein n=2.

11. A method for grouping a plurality of transformed chromatographic elution profiles obtained by the method of claim 1, said method for grouping comprising:
a) placing one or more markers, numbered 1 through i, each marker placed at a position where said transformed elution profiles show apparently clustered detector response values,
b) obtaining the first axis value and second axis value for each marker, each marker located on a boundary line perpendicular to said first axis,
c) for each marker, and along its associated boundary line, assigning a numerical grouping factor of n to the profiles that have a second axis value greater than the second axis value of said each marker, or otherwise assigning a grouping factor of 1 to the profiles, wherein n is an integer greater than 1,
d) for each profile, obtaining a total value comprising the sum of all the grouping factors assigned to said each profile,
e) grouping together those profiles having the same total value.

12. The method of claim 11 wherein n=2.

13. A system for transforming chromatographic elution profiles, said system comprising:
a computer having a processor and memory, wherein the computer receives a set of data corresponding to a plurality of chromatographic elution profiles, wherein each profile is obtained from the separation of a DNA mixture by Denaturing Matched Ion Polynucleotide Chromatography, wherein each DNA mixture comprises homoduplex and heteroduplex molecules obtained from the hybridization of a sample DNA and its corresponding wild type DNA, and wherein the processor:
a) overlays said profiles on a coordinate system comprising a first axis associated with time values and a second axis associated with detector response values,
b) selects first and second time points defining a time span wherein peaks due to said homoduplex and heteroduplex molecules are located within said span,
c) for each profile and within said span, adjusts the baseline by applying a slope factor to each detector response value, said factor derived from a line connecting the detector response values at said first and second time points, such that all of the profiles have a common baseline,
d) for each profile and within said span, normalizes the heights of the peaks to a pre-selected scale based on the height of the highest peak,
e) shifts of the profiles along said first axis such that all of the profiles intersect at a pre-selected point on the last eluting peak of each profile within said span.

14. The system of claim 13 wherein said pre-selected value is zero, wherein said pre-selected scale is from 0 to 1, wherein said pre-selected point comprises a point on the last eluting edge of said last eluting peak, and wherein in step (c) the second axis value at the first time point and the second axis value at the second time point are set to zero.

15. The system of claim 13 wherein said processor:
f) within said span, divides the first axis into a series of adjacent and evenly-spaced time regions wherein boundary lines, perpendicular to said first axis, are located between adjacent time regions,
g) divides each boundary line into a plurality of equal and adjacent segments,
h) for each boundary line, numbered 1 through i
i) determines the number of profiles intersecting each of said segments,
ii) determines the segment having the highest number of intersecting profiles and determines the nearest segment having zero intersecting profiles,
iii) for each boundary line, assigns a numerical grouping factor of nⁱ to the profiles that have a second axis value greater than said segment having zero intersecting profiles and assigns a grouping factor of 1 to the remaining intersecting profiles, wherein n is an integer greater than 1,
i) for each profile, obtains a total value comprising the sum of all the grouping factors assigned to said each profile,
j) groups together those profiles having the same total value.

16. The method of claim 15 wherein n=2.

17. The system of claim 15 wherein said processor
f) receives instructions for placing one or more markers, numbered 1 through i, each marker placed at a position where said transformed elution profiles show apparently clustered detector response values,
g) obtains the first axis value and second axis value for each marker, each marker located on a boundary line perpendicular to said first axis,
h) for each marker, and along its associated boundary line, assigns a numerical grouping factor of nⁱ to the profiles that have a second axis value greater than the second axis value of said each marker, or otherwise assigns a grouping factor of 1 to the profiles, wherein n is an integer greater than 1,
i) for each profile, obtains a total value comprising the sum of all the grouping factors assigned to said each profile,
j) groups together those profiles having the same total value.

18. The system of claim 17 wherein said processor applies one or more statistical criteria to the transformed profiles obtained after step (e) to determine whether or not to group said transformed profiles into a single group.

19. A computer readable medium for storing computer readable instructions, the instructions being capable of programming a computer to perform a method, the method comprising:
a method for transforming a plurality of chromatographic elution profiles, wherein each profile is obtained from the separation of a DNA mixture by Denaturing Matched Ion Polynucleotide Chromatography, wherein each DNA mixture comprises homoduplex and heteroduplex molecules obtained from the hybridization of a sample DNA and its corresponding wild type DNA, said method for transforming comprising:
a) overlaying said profiles on a coordinate system comprising a first axis showing time values and a second axis showing detector response values,
b) selecting first and second time points defining a time span wherein peaks due to said homoduplex and heteroduplex molecules are located within said span,
c) for each profile and within said span, adjusting the baseline by applying a slope factor to each detector response value, said factor derived from a line connecting the detector response values at said first and second time points, such that all of the profiles have a common baseline and the second axis value at the first time point and the second axis value at the second time point are set to zero,
d) for each profile and within said span, normalizing the heights of the peaks to a pre-selected scale based on the height of the highest peak,
e) shifting the profiles along said first axis such that all of the profiles intersect at a pre-selected point on the last eluting peak of each profile within said span.

20. A computer readable medium for storing computer readable instructions, the instructions being capable of programming a computer to perform a method, the method comprising:
the method for transforming of claim 19 and further comprising applying one or more statistical criteria to the transformed profiles obtained after step (e) to determine whether or not to group said transformed profiles into a single group.

21. A computer readable medium for storing computer readable instructions, the instructions being capable of programming a computer to perform a method, the method comprising:
the method for transforming of claim 19 and further comprising a method for grouping a plurality of transformed chromatographic elution profiles, said method for grouping comprising:
a) within said span, dividing the first axis into a series of adjacent and evenly-spaced time regions wherein boundary lines, perpendicular to said first axis, are located between adjacent time regions, wherein said profiles intersect said boundary lines,
b) for each boundary line and between the highest intersecting profile and the lowest intersecting profile, dividing said each boundary line into a plurality of equally spaced and adjacent segments,
c) for each boundary line, numbered 1 through i
i) determining the number of profiles intersecting each of said segments,
ii) determining the segment having the highest number of intersecting profiles and determining the nearest segment having zero intersecting profiles,
iii) for each boundary line, assigning a numerical grouping factor of nⁱ to the profiles that have a second axis value greater than said segment having zero intersecting profiles and assigning a grouping factor of 1 to the remaining intersecting profiles, wherein n is an integer greater than 1,
d) for each profile, obtaining a total value comprising the sum of all the grouping factors assigned to said each profile,
e) grouping together those profiles having the same total value.

22. The computer readable medium of claim 20 wherein n=2.

23. A computer readable medium for storing computer readable instructions, the instructions being capable of programming a computer to perform a method, the method comprising:
the method for transforming of claim 19 and further comprising a method for grouping a plurality of transformed chromatographic elution profiles, said method for grouping comprising:
a) placing one or more markers, numbered 1 through i, each marker placed at a position where said transformed elution profiles show apparently clustered detector response values,
b) obtaining the first axis value and second axis value for each marker, each marker located on a boundary line perpendicular to said first axis,
c) for each marker, and along its associated boundary line, assigning a numerical grouping factor of nⁱ to the profiles that have a second axis value greater than the second axis value of said each marker, or otherwise assigning a grouping factor of 1 to the profiles, wherein n is an integer greater than 1,
d) for each profile, obtaining a total value comprising the sum of all the grouping factors assigned to said each profile,
e) grouping together those profiles having the same total value.

24. The computer readable medium of claim 23 wherein n=2.

25. A plurality of transformed elution profiles obtained by the method of claim 1.

26. A plurality of elution profiles grouped by the method of claim 9.

27. A plurality of elution profiles grouped by the method of claim 11.
